SEQUENCE LISTING (73 sequences)

SEQ ID NO: 1 - DNA, 300 bp, Methylobacterium sp.
  source 1..300: mol_type = genomic DNA
acggtcaccccacggactgggcgagtacctcaccggtgttctatcataacgccgagttagttttcgaccgtcccttatgcgatgtaccaccggtgtcggcagccgatttcgtcccaccgggagctggcgttccggttcagaccaccatcatcggtcacgatgtctggattggacacggggccttcatctcccccggcgtgactataggaaacggcgcgatcgtcggggcccaggcggtcgtcacaagagatgtcccaccctatgcggtagttgctggcgtccccgcgaccgtacgacgat

SEQ ID NO: 2 - DNA, 300 bp, Methylobacterium sp.
  source 1..300: mol_type = genomic DNA
ccaataaaagcgttggccgcctgggcaacccgatccgagcctaagactcaaagcgcaagcgaacacttggtagagacagcccgccgactacggcgttccagcactctccggctttgatcggataggcattggtcaaggtgccggtggtgatgacctcgcccgccgcaagcggcgaattactcggatcagcggccagcacctcgaccaagtgtcggagcgcgaccaaagggccacgttcgaggacgtttgaggcgcgaccagtctcgatagtctcatcgtcgcggcgaagctgcacctcga

SEQ ID NO: 3 - DNA, 300 bp, Methylobacterium sp.
  source 1..300: mol_type = genomic DNA
cgatggcaccgacctgccatgcctctgccgtccgcgccagaatggtaaagaggacgaagggggtaaggatcgtcgctgcagtgttgagcagcgaccagagaagggggccgaacatcggcatcaaacctcgattgccactcggacgcgaagcgcgtcttgaaggagggatggaagcgaaacggccgcagagtaaccgccgacgaaagattgcacccctcatcgagcaggatcggaggtgaaggcaagcgtgggttattggtaagtgcaaaaaatataatggtagcgtcagatctagcgttc

SEQ ID NO: 4 - DNA, 23 bp, synthetic construct
  misc_feature 1..23: note = Synthetic
  source 1..23: mol_type = other DNA
cctcaccggtgttctatcataac

SEQ ID NO: 5 - DNA, 20 bp, synthetic construct
  misc_feature 1..20: note = Synthetic
  source 1..20: mol_type = other DNA
ccgatgatggtggtctgaac

SEQ ID NO: 6 - DNA, 21 bp, synthetic construct
  misc_feature 1..21: note = Synthetic
  source 1..21: mol_type = other DNA
cgtcccttatgcgatgtacca

SEQ ID NO: 7 - DNA, 22 bp, synthetic construct
  misc_feature 1..22: note = Synthetic
  source 1..22: mol_type = other DNA
gatccgagcctaagactcaaag

SEQ ID NO: 8 - DNA, 21 bp, synthetic construct
  misc_feature 1..21: note = Synthetic
  source 1..21: mol_type = other DNA
gaccaatgcctatccgatcaa

SEQ ID NO: 9 - DNA, 20 bp, synthetic construct
  misc_feature 1..20: note = Synthetic
  source 1..20: mol_type = other DNA
aacacttggtagagacagcc

SEQ ID NO: 10 - DNA, 21 bp, synthetic construct
  misc_feature 1..21: note = Synthetic
  source 1..21: mol_type = other DNA
aaggagggatggaagcgaaac

SEQ ID NO: 11 - DNA, 19 bp, synthetic construct
  misc_feature 1..19: note = Synthetic
  source 1..19: mol_type = other DNA
ataacccacgcttgccttc

SEQ ID NO: 12 - DNA, 21 bp, synthetic construct
  misc_feature 1..21: note = Synthetic
  source 1..21: mol_type = other DNA
cgcagagtaaccgccgacgaa

SEQ ID NO: 13 - DNA, 300 bp, Methylobacterium sp.
  source 1..300: mol_type = genomic DNA
gcaaaacgacctaatagttctacagcggcatgcgccaagtcagcgcggtgaacagtatacctgggagcaacttgtcctccgaaacccacataaaacaaattactcctggcagtgcccagtccatcaaaatcgaatacaatatttctcgaggaggcatctgtaatagcctgccaaagcaacaaagctatggcgccgttatgactttcattgcttctggtagacataaaataatatgccgatttgtgatcccaaatgtagaatattgccgcatcaattgcgccaagtttatttcggatcgat

SEQ ID NO: 14 - DNA, 300 bp, Methylobacterium sp.
  source 1..300: mol_type = genomic DNA
ggcgccaacggtatgatcgcatgattttcctgcggcatagcttgcgggaatggcgtatttggcgctctcctcaggaatttctaagggcatacgcaggaactctacagcacttttactggtattttgtagtgacagcggaggaggctggtgctcaaggtaatcgtgatgaagtgatccgggccattcggggcgcgtttctagtctttccaatccgcgccctgtaccacgtattacgccggaccggtctgcgccgcgccgccctcttgaccgccctaaatgtctaagagcgtctaacaaagc

SEQ ID NO: 15 - DNA, 300 bp, Methylobacterium sp.
  source 1..300: mol_type = genomic DNA
gacgatatcgctcatcttcactgcattgaagctggtgccgtactgcatagggatgaaaaagtgatgcggatagacggctgacgggaaagcgcctggtcgatcgaagactttgctgacgaggttgtggtagccccggatataggcatcgaaggccgggacgttgatcccatcctttgccttatcttgactggcgtcgtcgcgtgccgtcagaacgggcacgtcgcaggtcatcgaggccagcaccttgcggaacacctgcgttccgccgttgggattatcgacggcgaacgcggtggccgc

SEQ ID NO: 16 - DNA, 21 bp, synthetic construct
  misc_feature 1..21: note = Synthetic
  source 1..21: mol_type = other DNA
gtcctccgaaacccacataaa

SEQ ID NO: 17 - DNA, 23 bp, synthetic construct
  misc_feature 1..23: note = Synthetic
  source 1..23: mol_type = other DNA
ctaccagaagcaatgaaagtcat

SEQ ID NO: 18 - DNA, 22 bp, synthetic construct
  misc_feature 1..22: note = Synthetic
  source 1..22: mol_type = other DNA
tctgtaatagcctgccaaagca

SEQ ID NO: 19 - DNA, 19 bp, synthetic construct
  misc_feature 1..19: note = Synthetic
  source 1..19: mol_type = other DNA
ggctggtgctcaaggtaat

SEQ ID NO: 20 - DNA, 20 bp, synthetic construct
  misc_feature 1..20: note = Synthetic
  source 1..20: mol_type = other DNA
acatttagggcggtcaagag

SEQ ID NO: 21 - DNA, 19 bp, synthetic construct
  misc_feature 1..19: note = Synthetic
  source 1..19: mol_type = other DNA
atgaagtgatccgggccat

SEQ ID NO: 22 - DNA, 21 bp, synthetic construct
  misc_feature 1..21: note = Synthetic
  source 1..21: mol_type = other DNA
ccgtactgcatagggatgaaa

SEQ ID NO: 23 - DNA, 21 bp, synthetic construct
  misc_feature 1..21: note = Synthetic
  source 1..21: mol_type = other DNA
taaggcaaaggatgggatcaa

SEQ ID NO: 24 - DNA, 21 bp, synthetic construct
  misc_feature 1..21: note = Synthetic
  source 1..21: mol_type = other DNA
ttgctgacgaggttgtggtag

SEQ ID NO: 25 - DNA, 300 bp, Methylobacterium sp.
  source 1..300: mol_type = genomic DNA
caactatgtagacccgacggtgcgatttcacttcgcaaagccgcagggcagcacccttgcgctcaatgttgacgccagcgtgatctatactattaccgtcacgcacacgcagggcggcgtacagattcatcgcgagagtaagaaccaccatcagaccatcacgcgcagcgacctgagcaagcagttcggcgttggtgtggccgaccagctgacgcgcgatcaggtcatgaaggtgatcgagtcggcatttcgcgacgctacccgctaagatcggcgcccacgaaacgctacgagactagg

SEQ ID NO: 26 - DNA, 300 bp, Methylobacterium sp.
  source 1..300: mol_type = genomic DNA
agccggcatcttgttcaaggcgctcacctcgacgccgacgctgtaggcgacttgagagggcgtctcatatgaacgaagcatcttcgcgtagagaaccttcttgttctcctgcgtgatgttcgctttgcagacgttgactgccgccatgaacgccgaagccttgcgcgcttcatcgtaatcgcctgcgaaggcgggtagtgaaaagcttagtgcaatggcaaacacagccgccgaacgtcgcatggtatccgtccccgattgacggcagtgccgccatatctcggctttagcagagctgat

SEQ ID NO: 27 - DNA, 300 bp, Methylobacterium sp.
  source 1..300: mol_type = genomic DNA
aacctgcgccggccgaggtttcgcgagccgtcgccacgggcaacgcctcgcccgcgatgtgcaaaaaagtccccggcacttcgcgccgtcgtccgatccacgaccgcgaatttctcaacgagtacaaggtgcttatgggagatccgagcgtccgtcccggagcccgagaccgcgcggcccgagtaataggcgaaaaagactcctactcctcgggcttctcgggccccctcagcaacatctacgcttgccgcccatcaccctggcgggagatcagcgacgagacacaggcccacttcgccc

SEQ ID NO: 28 - DNA, 22 bp, synthetic construct
  misc_feature 1..22: note = Synthetic
  source 1..22: mol_type = other DNA
ttgacgccagcgtgatctatac

SEQ ID NO: 29 - DNA, 22 bp, synthetic construct
  misc_feature 1..22: note = Synthetic
  source 1..22: mol_type = other DNA
gtgatggtctgatggtggttct

SEQ ID NO: 30 - DNA, 19 bp, synthetic construct
  misc_feature 1..19: note = Synthetic
  source 1..19: mol_type = other DNA
tattaccgtcacgcacacg

SEQ ID NO: 31 - DNA, 24 bp, synthetic construct
  misc_feature 1..24: note = Synthetic
  source 1..24: mol_type = other DNA
cttcgcgtagagaaccttcttgtt

SEQ ID NO: 32 - DNA, 22 bp, synthetic construct
  misc_feature 1..22: note = Synthetic
  source 1..22: mol_type = other DNA
cttcgcaggcgattacgatgaa

SEQ ID NO: 33 - DNA, 20 bp, synthetic construct
  misc_feature 1..20: note = Synthetic
  source 1..20: mol_type = other DNA
cgtgatgttcgctttgcaga

SEQ ID NO: 34 - DNA, 23 bp, synthetic construct
  misc_feature 1..23: note = Synthetic
  source 1..23: mol_type = other DNA
ccgcgaatttctcaacgagtaca

SEQ ID NO: 35 - DNA, 21 bp, synthetic construct
  misc_feature 1..21: note = Synthetic
  source 1..21: mol_type = other DNA
gcccgaggagtaggagtcttt

SEQ ID NO: 36 - DNA, 20 bp, synthetic construct
  misc_feature 1..20: note = Synthetic
  source 1..20: mol_type = other DNA
aggtgcttatgggagatccg

SEQ ID NO: 37 - DNA, 300 bp, Methylobacterium sp.
  source 1..300: mol_type = genomic DNA
agtcattgatcaagcaacccctattgagttggatatcgaaggatcaaggtcgcgtcaatagatgcatctatcaggccaaatgtcgcttttcaagaatggctctttcgaagctatctttataatcgctcgccattctctcattaccaaaatcgaccttaactagctcgacattgatgcgagcagctccggcaaacgaggagagattgaccttaaaggaattgaacgcctcaagcaattcagacacattaccaggagtgctatagcaacaaccagacccatatcggtcaataacctctttta

SEQ ID NO: 38 - DNA, 300 bp, Methylobacterium sp.
  source 1..300: mol_type = genomic DNA
cgcaaaacgatttatcactgccatcttgttgtttgataacccttttttaccagacgttatgctgggcgagaaagaggactagcagatcggagcggtatcgcgatttttcggtagttcgcgcctacaacaggataagatccgatagtgaagcaacatggctgttttttgatttgtaagtcagcaacttaagcagccagcctatctgccgtcgcagacgcttgaggcatcgggcagcatcttagaaaaggtggcagtaattgccacagcggaacgtagcggcacggataagcacgcagggtc

SEQ ID NO: 39 - DNA, 300 bp, Methylobacterium sp.
  source 1..300: mol_type = genomic DNA
cccatctggacccaatatccccttcatcgacaattcccgagtaagtgtgggttcgaggatttcgcgaaacagccttgttcgttcctccggccttaaaattggcgtgccgtcgggagatcgataggcatcccttacctgcctttcgaccgccggcacacgcgcgccggtcgtcgtgttcacggccacggaatggacgaaggtgcgccgctcatttcgctcgtttgccgtctccaccatccaggaggccagcaggacggtttcgtctcgaccgccggtcacacacaccgcaagggactcagg

SEQ ID NO: 40 - DNA, 22 bp, synthetic construct
  misc_feature 1..22: note = Synthetic
  source 1..22: mol_type = other DNA
tcgctcgccattctctcattac

SEQ ID NO: 41 - DNA, 22 bp, synthetic construct
  misc_feature 1..22: note = Synthetic
  source 1..22: mol_type = other DNA
aggtcaatctctcctcgtttgc

SEQ ID NO: 42 - DNA, 18 bp, synthetic construct
  misc_feature 1..18: note = Synthetic
  source 1..18: mol_type = other DNA
tcgacattgatgcgagca

SEQ ID NO: 43 - DNA, 22 bp, synthetic construct
  misc_feature 1..22: note = Synthetic
  source 1..22: mol_type = other DNA
ttcgcgcctacaacaggataag

SEQ ID NO: 44 - DNA, 23 bp, synthetic construct
  misc_feature 1..23: note = Synthetic
  source 1..23: mol_type = other DNA
cagataggctggctgcttaagtt

SEQ ID NO: 45 - DNA, 18 bp, synthetic construct
  misc_feature 1..18: note = Synthetic
  source 1..18: mol_type = other DNA
tccgatagtgaagcaaca

SEQ ID NO: 46 - DNA, 24 bp, synthetic construct
  misc_feature 1..24: note = Synthetic
  source 1..24: mol_type = other DNA
gagtaagtgtgggttcgaggattt

SEQ ID NO: 47 - DNA, 23 bp, synthetic construct
  misc_feature 1..23: note = Synthetic
  source 1..23: mol_type = other DNA
aggtaagggatgcctatcgatct

SEQ ID NO: 48 - DNA, 18 bp, synthetic construct
  misc_feature 1..18: note = Synthetic
  source 1..18: mol_type = other DNA
cggaggaacgaacaaggc

SEQ ID NO: 49 - DNA, 300 bp, Methylobacterium sp.
  source 1..300: mol_type = genomic DNA
acctgctaaaatcacgtcctctcagattgaaaaatcattgaagaaacgtgtcgaacgattgccggggattatgacgttagatcaattgaaaaatacaagctttgaaattgagttacagccaaaagatgccccggatccggacccatcagacttcggtggctagttcgagccaaactcgaacgtcgccatggcgcgcaagtcgcaataccatttcacagcgcagcggttatttcgttgtacactgtagcaatgcgtcggcttgcgcgcttccgctggcgatcaaaggtccgccgatttacg

SEQ ID NO: 50 - DNA, 300 bp, Methylobacterium sp.
  source 1..300: mol_type = genomic DNA
tcccgaacatacaatggaggaagcgtgtggtaggccaatttgtaacgaaatatggcatcggtcacggctctctcaataaattcgatctcaagtcttctgaacgagcatgcctcatccttatcctgagcgaacgcctgccagtttgcagtcattccaacatacatagccaaaaaggcgaggtagaccttcatacgggcacctcaatcgtccccattcgttcaagctccttcaagataacagccgcaccacattgctgagatcgaagattcggatcaaatattccatcaaatttatactttc

SEQ ID NO: 51 - DNA, 300 bp, Methylobacterium sp.
  source 1..300: mol_type = genomic DNA
gcatcctttgcgctcgcaggcctaaggtcaagcccggttacttcgtttggtagaacgaggtagacgatgcctagtcttaaggtggcccatgttaaccaacagggccagaacatgattatagttccgttagatgccaacttcggttacaaaaccgatggtgagcagtccgacatcatgttcgaaatacaggacgcggcgcggtccgccggtcttgcgggtgccgtagtagcgttctggcagtcaggtggacaaacccgtttccggggcccggctccgtggcacccattccttcgcagcctc

SEQ ID NO: 52 - DNA, 22 bp, synthetic construct
  misc_feature 1..22: note = Synthetic
  source 1..22: mol_type = other DNA
gcgcaagtcgcaataccatttc

SEQ ID NO: 53 - DNA, 21 bp, synthetic construct
  misc_feature 1..21: note = Synthetic
  source 1..21: mol_type = other DNA
cgtaaatcggcggacctttga

SEQ ID NO: 54 - DNA, 19 bp, synthetic construct
  misc_feature 1..19: note = Synthetic
  source 1..19: mol_type = other DNA
cgcagcggttatttcgttg

SEQ ID NO: 55 - DNA, 22 bp, synthetic construct
  misc_feature 1..22: note = Synthetic
  source 1..22: mol_type = other DNA
acgagcatgcctcatccttatc

SEQ ID NO: 56 - DNA, 21 bp, synthetic construct
  misc_feature 1..21: note = Synthetic
  source 1..21: mol_type = other DNA
cgattgaggtgcccgtatgaa

SEQ ID NO: 57 - DNA, 20 bp, synthetic construct
  misc_feature 1..20: note = Synthetic
  source 1..20: mol_type = other DNA
tgccagtttgcagtcattcc

SEQ ID NO: 58 - DNA, 23 bp, synthetic construct
  misc_feature 1..23: note = Synthetic
  source 1..23: mol_type = other DNA
cccggttacttcgtttggtagaa

SEQ ID NO: 59 - DNA, 24 bp, synthetic construct
  misc_feature 1..24: note = Synthetic
  source 1..24: mol_type = other DNA
cgaagttggcatctaacggaacta

SEQ ID NO: 60 - DNA, 20 bp, synthetic construct
  misc_feature 1..20: note = Synthetic
  source 1..20: mol_type = other DNA
tggcccatgttaaccaacag

SEQ ID NO: 61 - DNA, 300 bp, Methylobacterium sp.
  source 1..300: mol_type = genomic DNA
gcccttctgtcaggcgatattgtataatggcgttgccccaatagaagcagccattcgtgcgagggcagcagcgacgctaggtcgaaagagcatcctaatctcgatcaagatgcgactgagatttctgatgaaaatatctagacacaagcaaagctggtgaaattacaacgatcatggcgacaattgcggccaattcggccggaacttgaaggaacataaaaatgaatattacaaatataccgcaaagcatgtagagttgctacaccaagggtcgggacgtccaaaaaaactcactgagga

SEQ ID NO: 62 - DNA, 300 bp, Methylobacterium sp.
  source 1..300: mol_type = genomic DNA
ggaacataaaaatgaatattacaaatataccgcaaagcatgtagagttgctacaccaagggtcgggacgtccaaaaaaactcactgaggaagtcgactggaagcacgaggcgccccccccaggagcggggcgaccggcaagggggcccgcaattgtcgccatgatcgaccagcttaggtaggatcctctttcgacctaacgaatggctgcttctattggggcaacgccattatacaatatcgcctgaccatctggaacgcggcccggtccaccggcaggttggcgacgacagcgtcggag

SEQ ID NO: 63 - DNA, 300 bp, Methylobacterium sp.
  source 1..300: mol_type = genomic DNA
cggcgtcgaccagccgggcgaactgcttgggcatgctctcccgcgacgccggccacagccgcgtccccgtccctccgcacaggatcatcgggtggatttgaaaggcaaaacgggacatcaggataggccgctcaggcgttggcgctgaggcgcttgatgtcggcgtcgaccatctcggtgatcagcgcctcgaggctggtctcggcctcccagccgaaggtcgccttggccttggcggggttgcccagcagcacctcgacctctgccggccggaacagcgccgggtcgacgatcaggtgg

SEQ ID NO: 64 - DNA, 300 bp, Methylobacterium sp.
  source 1..300: mol_type = genomic DNA
ctggacatgcgcccaccccggccaagtccgaccgcaccggcaaccgctcctgtagtcgtcgtcatcgttctcacccctgaggcggagaccgtccgctaacggggtgtctcaagcaaccgtggggcggaggaacacgcacgtagtcgcgtttcaaggttcgcacgaacgcctcggccatgccgttgctctgcgggctctccagcggcgtcgtttttggcaccaaaccaaggtcgcgggcgaagcggcgcgtgtcgcggggactgtcaggaatttcgtgtgggggcggccatagtggatccg

SEQ ID NO: 65 - DNA, 300 bp, Methylobacterium sp.
  source 1..300: mol_type = genomic DNA
ggaaatcggcttcaagtacgacgtcacgccggccatgcaggtcacgggtgcactgttcaatctcgagcgcgacaaccagccgttcccctcgaacgtggagtccggcctcgtccttggcgcaggtcagacacgcacccagggcgcggaaatcggcctggccggctatctaaccgattggtggcaggtctttggcggctacgcttataccgaggcacgcgtactctcgccactggaagacgatggagacgtgatcgcagcaggtaatctcgtcggcaacgttccgctaaatactttcagtct

SEQ ID NO: 66 - DNA, 300 bp, Methylobacterium sp.
  source 1..300: mol_type = genomic DNA
cggcctggccggctatctaaccgattggtggcaggtctttggcggctacgcttataccgaggcacgcgtactctcgccactggaagacgatggagacgtgatcgcagcaggtaatctcgtcggcaacgttccgctaaatactttcagtctgttcaacaagttcgatatcaacgagaatttctccgttgctctgggctattactatcaggatgccagctttgcctcctcagacaatgcagtgcgtttgccaagttattcgcggttcgatggcgggttgttctatcgattcgacgagttgac

SEQ ID NO: 67 - DNA, 300 bp, Methylobacterium sp.
  source 1..300: mol_type = genomic DNA
acgttccgctaaatactttcagtctgttcaacaagttcgatatcaacgagaatttctccgttgctctgggctattactatcaggatgccagctttgcctcctcagacaatgcagtgcgtttgccaagttattcgcggttcgatggcgggttgttctatcgattcgacgagttgacacgcgttcagcttagcgtcgagaacattttcgacaggcgttacatcatcaactccaacaacaacaacaacctcacgcctggcgcgccgagaacagtccgcgtgcaattgatcgctcggttctaaa

SEQ ID NO: 68 - DNA, 300 bp, Methylobacterium sp.
  source 1..300: mol_type = genomic DNA
agcccacaagcctgatgcacttaactacatcctctaatgtcgcgccaatttgcttggcggcaggggatgttgtatcgtcataggcttgtctaaccggaacttgtttgccaatctctttggcgatcgcaaccgccatctcgtgttcgtcaaccatgtgcgcgttcctctaattgcactcatggtgccacgtgcacctccgatcgtctcgtgtctagaatgaaggtgggaacaaccttacacaggctttcgcgacgcgcgaatttctggtttctccgcctcggatgtgggtttgagcgcttc

SEQ ID NO: 69 - DNA, 300 bp, Methylobacterium sp.
  source 1..300: mol_type = genomic DNA
cttttcatttgtcatgatctcgaccaaggtattcacggcaagctcggtctgttgcttagcaagtgcctgaacttcgcgaacgatcggctctcgacccttcgggttcgagacctgtcccttttgaaaaccacgtgccctacacttttcgggatcaaggtgcgggttggctttggtcaaaattctctggcgtcccattacacgccctccgcatcatcgttcccgcgaacgatctgacccccgacttccgcgaggaagcgtgtggcgtgatcctcgaagcggaatgccacctcgaactgttcc

SEQ ID NO: 70 - DNA, 300 bp, Methylobacterium sp.
  source 1..300: mol_type = genomic DNA
cagcagcaagcagatcgttgaaaaccgcttgaaccgcatcttgatcgggaccggaaccaatcaggtcatctaggtaaaccgagacgtaaactcgtttgcgctcggcatctttcagaacgtccgtgatgccagaccgcattagtaccatcgtcgccaaggcgggcgactgaacgaagccgatcggcagagagtaacggggaccgcccctaatcgggttgcgaacgcaagaccacttagcaaaggttcgagcacggccgaacttcgcatggtggagagccgcggcaacacggttccgtgata

SEQ ID NO: 71 - DNA, 300 bp, Methylobacterium sp.
  source 1..300: mol_type = genomic DNA
tagacattccaacaaaccggcaagaggctcgtcctcactcgaggatttgttgggacttgcatgatgtcgaagcggagccgttatgacctgggtgcgatcatgcgccgagcatgggagatggctcgggaggcggcattcgcggttggcgagcgggcacggactcaccttgctgccgcgatgcgcagcgcgtgggccgaagccaagttggcactcgcgcccacgaagacggagcaggatcgtctctctccgagcgacatgatcggacatgaggacgcctaccaaggccgggttctaaaatat

SEQ ID NO: 72 - DNA, 300 bp, Methylobacterium sp.
  source 1..300: mol_type = genomic DNA
aagatggatacgacaagcgcgattacattatttgcgaaatagatggacaaataaaagacaaaggactgatgtatttccttaaatctggacaagttgacctctttcacatagaagtcaccactccctttgggacaatttggtgtcacgaaaacatagaggccgaacttcttagctgaattatcgcgctccgggttcttatgcggctgagtgaagcgcgggacagcttgcgagcagggccgccaatggcagccgggatgacacaatgctcggtctcccgacgcttcttcaatcgggagcgct

SEQ ID NO: 73 - DNA, 299 bp, Methylobacterium sp.
  source 1..299: mol_type = genomic DNA
agctgaattatcgcgctccgggttcttatgcggctgagtgaagcgcgggacagcttgcgagcagggccgccaatggcagccgggatgacacaatgctcggtctcccgacgcttcttcaatcgggagcgcttcgcagcccggggcggcgcgctcatgcgtcacgacctgggccctgcgcaccttcgcggccccgccgtcccggcagatccctgatgccccaagtgggcggccactccatcaaagaaccccggcctgtggcagatctcgtaggcataccgaggttccgcagtgcccccacc

(19) United States
(12) Patent Application Publication
(10) Pub. No.: US 2025/0241315 A1
(43) Pub. Date: Jul. 31, 2025

(54) METHYLOBACTERIUM COMPOSITIONS FOR IMPROVING CORN YIELD

(71) Applicant: NewLeaf Symbiotics, Inc., St. Louis, MO (US)

(72) Inventors: Natalie Breakfield; Charles Michael McFatrich, St. Louis, MO (US)

(21) Appl. No.: 19/031,518

(22) Filed: Jan. 18, 2025

Related U.S. Application Data
(63) Continuation of application No. 17/299,409, filed on Jun. 3, 2021.
(60) Provisional application No. 62/774,640, filed on Dec. 3, 2018.
(60) Provisional application No. 62/878,164, filed on Jul. 24, 2019.

(51) Int. Cl. A01N 63/20; A01N 25/04; A01N 25/12; C12Q 1/689

(52) U.S. Cl. CPC A01N 63/20; A01N 25/04; A01N 25/12; C12Q 1/689

(57) ABSTRACT

The present invention provides yield enhancing compositions comprising Methylobacterium, methods for improving corn yield, and methods of making the compositions. Also provided are isolated yield enhancing Methylobacterium.

### CROSS-REFERENCE TO RELATED APPLICATIONS

This patent application is a continuation of U.S. patent application Ser. No. 17/299,409 filed Jun. 3, 2021, which is a 371 U.S. National phase application of International Application No. PCY/US2019/64034 filed Dec. 2, 2019, which claims the benefit of U.S. Patent Application No. 62/774,640, filed Dec. 3, 2018, and U.S. Patent Application No. 62/878, 164, filed Jul. 24, 2019, which are each incorporated herein by reference in their entireties.

### SEQUENCE LISTING STATEMENT

A sequence listing containing the file named “NLSYM9796USN2 Sequence Listing.xml” which is 90,248 bytes (measured in MS-Windows®) and created on Mar. 31, 2025, contains 73 nucleotide sequences, is provided herewith, and is incorporated herein by reference in its entirety.

### BACKGROUND

One-carbon organic compounds such as methane and methanol are found extensively in nature, and are utilized as carbon sources by bacteria classified as methanotrophs and methylotrophs. Methanotrophic bacteria include species in the genera Methylobacter, Methylomonas, Methylomicrobium, Methylococcus, Methylosinus, Methylocystis, Methylosphaera, Methylocaldum, and Methylocella (Lidstrom, 2006). Methanotrophs possess the enzyme methane monooxygenase, that incorporates an atom of oxygen from O₂ into methane, forming methanol. All methanotrophs are obligate one-carbon utilizers that are unable to use compounds containing carbon-carbon bonds. Methylotrophs, on the other hand, can also utilize more complex organic compounds, such as organic acids, higher alcohols, sugars, and the like. Thus, methylotrophic bacteria are facultative methylotrophs. Methylotrophic bacteria include species in the genera Methylobacterium, Hyphomicrobium, Methylophilus, Methylobacillus, Methylophaga, Aminobacter, Methylorhabdus, Methylopila, Methylosulfonomonas, Marinosulfonomonas, Paracoccus, Xanthobacter, Ancylobacter (also known as Microcyclus), Thiobacillus, Rhodopseudomonas, Rhodobacter, Acetobacter, Bacillus, Mycobacterium, Arthobacter, and Nocardia (Lidstrom, 2006).

Most methylotrophic bacteria of the genus Methylobacterium are pink-pigmented. They are conventionally referred to as PPFM bacteria, being pink-pigmented facultative methylotrophs. Green (2005, 2006) identified twelve validated species in the genus Methylobacterium, specifically M. aminovorans, M. chloromethanicum, M. dichloromethanicum, M. extorquens, M. fujisawaense, M. mesophilicum, M. organophilum, M. radiotolerans, M. rhodesianum, M. rhodinum, M. thiocyanatum, and M. zatmanii. However, M. nidulans is a nitrogen-fixing Methylobacterium that is not a PPFM (Sy et al., 2001). Methylobacterium are found in soil, dust, fresh water, sediments, and leaf surfaces, as well as in industrial and clinical environments (Green, 2006).

### SUMMARY

Methods for improving corn plant yield that comprise: (a) applying a composition a to a corn plant or a corn plant part, wherein the composition comprises (i) Methylobacterium isolate NLS0807 (NRRL B-67743), NLS0662 (NRRL B-67742), NLS0648 (NRRL B-67741), NLS0109 (NRRL B-67340), or variants thereof, or (ii) a combination of Methylobacterium isolate NLS0109 (NRRL B-67340) or a variant thereof, and Methylobacterium isolate NLS0017 (B-50931) or a variant thereof; wherein said composition further comprises at least one additional component selected from the group consisting of an additional active ingredient, an agriculturally acceptable adjuvant, and an agriculturally acceptable excipient; and, (b) growing the corn plant to maturity, thereby improving yield of the corn plant are provided herein. In certain embodiments of the methods, the composition is applied to a corn seed. In certain embodiments of the aforementioned methods, the composition comprises a solid substance with the Methylobacterium grown thereon and adhered thereto, or an emulsion having the Methylobacterium grown therein. In certain embodiments of any of the aforementioned methods, the composition comprises the Methylobacterium at a titer of about 1×10⁶ CFU/gm to about 1×10¹⁴ CFU/gm for a solid composition or at a titer of about 1×10⁶ CFU/mL to about 1×10¹¹ CFU/mL for a liquid composition. In certain embodiments of the aforementioned methods, the composition comprises NLS0807 (NRRL B-67743), NLS0662 (NRRL B-67742), or a variant thereof. In certain embodiments of any of the aforementioned methods, the composition comprises NLS0109 or a variant thereof and NLS0017 or a variant thereof. In certain embodiments of any of the aforementioned methods, the Methylobacterium variant is glyphosate resistant or glufosinate resistant. In certain embodiments of the aforementioned methods, the applied composition coats or partially coats the corn plant or a part thereof. In certain embodiments of the aforementioned methods, the composition is applied to foliage of the corn plant. In certain embodiments of the aforementioned methods, the composition further comprises a fungicide. In certain embodiments of the aforementioned methods, the composition is applied at about the VE to about the V3 stage of development, about the V3 to about the V5 stage of development, about the V2 to V4, or V3 stage of development. In certain embodiments of the aforementioned methods, the methods further comprise the step of harvesting seed from the mature corn plant. In certain embodiments of the aforementioned methods, yield of harvested seed is increased in comparison to yield of harvested seed obtained from a control corn plant that did not receive an application of the Methylobacterium. In certain embodiments of the aforementioned methods, the composition is applied by spraying, coating, partially coating, immersing, and/or imbibing the corn plant or plant part with the composition. In certain embodiments of the aforementioned methods, the applied composition coats or partially coats the corn plant or a part thereof, wherein partial coating includes coating at least 5%, 10%, 20%, 30%, 40%, 50%, 60%, 70%, 80%, 90%, 95%, 98%, 99%, or about 99.5% of the surface area of the corn plant or a part thereof. In certain embodiments of the aforementioned methods, the corn plant part is a seed. In certain embodiments of the aforementioned methods, the composition comprises Methylobacterium isolate NLS0807 (NRRL B-67743), NLS0662 (NRRL B-67742), NLS0648 (NRRL B-67741), NLS0109 (NRRL B-67340), or a combination of Methylobacterium isolates NLS0109 (NRRL B-67340) and NLS0017 (B-50931). In certain embodiments of the aforementioned methods, the composition further comprises an additional active ingredient. In certain embodiments of the aforementioned methods, the additional active ingredient is selected from the group consisting of a fungicide, insecticide, nematicide, and biological. In certain embodiments of the aforementioned methods, the biological is a biocontrol agent other than NLS0109. In certain embodiments of the aforementioned methods, the additional active ingredient is selected from the group consisting of clothianidin, Bacillus firmus, abamectin, thiamethoxam, imidacloprid, azoxystrobin, fluopyram, fluoxastrobin, ipconazole, mefenoxam, metalaxyl, penflufen, prothioconazole, pyraclostrobin, and sedaxane. In certain embodiments of the aforementioned methods, the growing of the corn plant occurs in a field in the continental United States located east of the Mississippi river. In certain embodiments of the aforementioned methods, the growing of the corn plant occurs in a field in the continental United States located west of the Mississippi river. In certain embodiments of the aforementioned methods, the composition comprises (i) a Methylobacterium wherein the chromosomal genomic DNA has at least 99%, 99.9, 99.8, 99.7, 99.6%, or 99.5% sequence identity to chromosomal genomic DNA of NLS0807 (NRRL B-67743), NLS0662 (NRRL B-67742), NLS0648 (NRRL B-67741), or NLS0109 (NRRL B-67340), or (ii) a combination of Methylobacterium isolates wherein the chromosomal genomic DNA of said isolates has at least 99%, 99.9, 99.8, 99.7, 99.6%, or 99.5% sequence identity to chromosomal genomic DNA of NLS0109 (NRRL B-67340) or NLS0017 (B-50931). In certain embodiments, the composition comprises (i) a Methylobacterium having genomic DNA comprising one or more polynucleotide marker fragments of at least 50, 60, 100, 120, 180, 200, 240, or 300 nucleotides of SEQ ID NOS: 25-27, SEQ ID NOS: 37-39, SEQ ID NOS: 49-51 or SEQ ID NOS: 1-3; or (ii) a combination of Methylobacterium isolates wherein a first isolate has genomic DNA comprising one or more polynucleotide marker fragments of at least 50, 60, 100, 120, 180, 200, 240, or 300 nucleotides of SEQ ID NOS: 1-3 and a second isolate has genomic DNA comprising one or more polynucleotide marker fragments of at least 50, 60, 100, 120, 180, 200, 240, or 300 nucleotides of SEQ ID NOS: 13-15. In certain embodiments, the Methylobacterium has in its genome one or more marker fragments comprising a sequence having at least 98%, 99%, or 99.5% sequence identity across the entire length of SEQ ID NOS: 9-11. In certain embodiments of the aforementioned methods, the composition further comprises a second biological selected from the group consisting of ISO01 (NRRL B-50929), ISO02 (NRRL B-50930), ISO03 (NRRL B-50931), ISO04 (NRRL B-50932), ISO05 (NRRL B-50933), ISO06 (NRRL B-50934), ISO07 (NRRL B-50935), ISO08 (NRRL B-50936), ISO09 (NRRL B-50937), ISO10 (NRRL B-50938), ISO11 (NRRL B-50939), ISO12 (NRRL B-50940), ISO13 (NRRL B-50941), and ISO14 (NRRL B-50942).

Also provided are corn plants or corn plant parts that are coated or partially coated with a composition comprising a Methylobacterium, wherein the Methylobacterium is (i) NLS0807 (NRRL B-67743), NLS0662 (NRRL B-67742), NLS0648 (NRRL B-67741), or variants thereof, or (ii) a combination of Methylobacterium isolate NLS0109 (NRRL B-67340) or a variant thereof, and Methylobacterium isolate NLS0017 (B-50931) or a variant thereof. In certain embodiments, the composition further comprises at least one second component selected from the group consisting of an additional active ingredient, an agriculturally acceptable adjuvant, and an agriculturally acceptable excipient. In certain aforementioned embodiments, the composition comprises the Methylobacterium at a titer of about 1×10⁶ CFU/gm to about 1×10¹⁴ CFU/gm for a solid composition or at a titer of about 1×10⁶ CFU/mL to about 1×10¹¹ CFU/mL for a liquid composition. In certain aforementioned embodiments, Methylobacterium is Methylobacterium isolate NLS0807 (NRRL B-67743) or a variant thereof. In certain aforementioned embodiments, the corn plant part is selected from the group consisting of a seed, a leaf, an ear, and a tassel. In certain aforementioned embodiments, the composition comprises Methylobacterium isolate NLS0807 (NRRL B-67743), NLS0662 (NRRL B-67742), NLS0648 (NRRL B-67741), or a combination of Methylobacterium isolates NLS0109 (NRRL B-67340) and NLS0017 (B-50931). In certain aforementioned embodiments, the composition comprises an additional active ingredient. In certain aforementioned embodiments, the additional active ingredient is selected from the group consisting of a fungicide, insecticide, nematicide, and biological. In certain aforementioned embodiments, the biological is a biocontrol agent other than NLS0109. In certain aforementioned embodiments, the additional active ingredient is selected from the group consisting of clothianidin, Bacillus firmus, abamectin, thiamethoxam, imidacloprid, azoxystrobin, fluopyram, fluoxastrobin, ipconazole, mefenoxam, metalaxyl, penflufen, prothioconazole, pyraclostrobin, and sedaxane. In certain aforementioned embodiments, the composition comprises (i) a Methylobacterium wherein the chromosomal genomic DNA has at least 99%, 99.9, 99.8, 99.7, 99.6%, or 99.5% sequence identity to chromosomal genomic DNA of NLS0807 (NRRL B-67743), NLS0662 (NRRL B-67742), NLS0648 (NRRL B-67741), or NLS0109 (NRRL B-67340), or (ii) a combination of Methylobacterium isolates wherein the chromosomal genomic DNA of said isolates has at least 99%, 99.9, 99.8, 99.7, 99.6%, or 99.5% sequence identity to chromosomal genomic DNA of NLS0109 (NRRL B-67340) or NLS0017 (B-50931). In certain aforementioned embodiments, the composition further comprises a second biological selected from the group consisting of ISO01 (NRRL B-50929), ISO02 (NRRL B-50930), ISO03 (NRRL B-50931), ISO04 (NRRL B-50932), ISO05 (NRRL B-50933), ISO06 (NRRL B-50934), ISO07 (NRRL B-50935), ISO08 (NRRL B-50936), ISO09 (NRRL B-50937), ISO10 (NRRL B-50938), ISO11 (NRRL B-50939), ISO12 (NRRL B-50940), ISO13 (NRRL B-50941), and ISO14 (NRRL B-50942).

### DESCRIPTION

### Definitions

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Where a term is provided in the singular, embodiments comprising the plural of that term are also provided.

As used herein, the terms “include,” “includes,” and “including” are to be construed as at least having the features or encompassing the items to which they refer while not excluding any additional unspecified features or unspecified items.

As used herein, the phrases “adhered thereto” and “adherent” refer to Methylobacterium that are associated with a solid substance by growing, or having been grown, on a solid substance.

As used herein, the phrase “active ingredient” refers to a biological or pesticide in a composition for treatment of plants and/or plant parts.

As used herein, the term “biological” refers to a component of a composition for treatment of plants or plant parts comprised of or derived from a microorganism. Biologicals include biocontrol agents, other beneficial microorganisms, microbial extracts, natural products, plant growth activators or plant defense agents. Non-limiting examples of biocontrol agents include bacteria, fungi, beneficial nematodes, and viruses.

As used herein, the phrase “agriculturally acceptable adjuvant” refers to a substance that enhances the performance of a biological or pesticide in a composition for treatment of plants and/or plant parts. In certain compositions, a biological can comprise a mono-culture or co-culture of Methylobacterium.

As used herein, the phrase “agriculturally acceptable excipient” refers to an essentially inert substance that can be used as a diluent and/or carrier for a biological or pesticide in a composition for treatment of plants and/or plant parts. In certain compositions, a biological can comprise a mono-culture or co-culture of Methylobacterium.

As used herein, the term “strain” shall include all isolates of such strain.

As used herein, the phrase “control plant” refers to a plant that had not received treatment with a yield enhancing Methylobacterium or composition comprising the same at either the seed or any subsequent stage of the control plant's development. In certain embodiments, a control plant can be a plant that was treated with an additional active ingredient or a yield neutral Methylobacterium.

As used herein, the phrase “co-culture of Methylobacterium” refers to a Methylobacterium culture comprising at least two strains of Methylobacterium or at least two species of Methylobacterium.

As used herein, the phrase “contaminating microorganism” refers to microorganisms in a culture, fermentation broth, fermentation broth product, or composition that were not identified prior to introduction into the culture, fermentation broth, fermentation broth product, or composition.

As used herein, “variant” when used in the context of a Methylobacterium isolate, refers to any isolate that has chromosomal genomic DNA with at least 99%, 99.9, 99.8, 99.7, 99.6%, or 99.5% sequence identity to chromosomal genomic DNA of a deposited Methylobacterium isolate provided herein. A variant of an isolate can be obtained from various sources including soil, plants or plant material, and water, particularly water associated with plants and/or agriculture. Variants include derivatives obtained from deposited isolates. Methylobacterium isolates or strains can be sequenced (for example as taught by Sanger et al. (1977), Bentley et al. (2008) or Caporaso et al. (2012)) and genome-scale comparison of the sequences conducted (Konstantinos et al. (2005)) using sequence analysis tools (for example, BLAST, as taught by Altschul et al. (1990)).

As used herein, “derivative” when used in the context of a Methylobacterium isolate, refers to any Methylobacterium that is obtained from a deposited Methylobacterium isolate provided herein. Derivatives of a Methylobacterium isolate include, but are not limited to, derivatives obtained by selection, derivatives selected by mutagenesis and selection, and genetically transformed Methylobacterium obtained from a Methylobacterium isolate. A “derivative” can be identified, for example based on genetic identity to the strain or isolate from which it was obtained and will generally exhibit chromosomal genomic DNA with at least 99%, 99.9, 99.8, 99.7, 99.6%, or 99.5% sequence identity to chromosomal genomic DNA of the strain or isolate from which it was derived.

As used herein, the term “emulsion” refers to a colloidal mixture of two immiscible liquids wherein one liquid is the continuous phase and the other liquid is the dispersed phase. In certain embodiments, the continuous phase is an aqueous liquid and the dispersed phase is liquid that is not miscible, or partially miscible, in the aqueous liquid.

As used herein, the phrase “essentially free of contaminating microorganisms” refers to a culture, fermentation broth, fermentation product, or composition where at least about 95% of the microorganisms present by amount or type in the culture, fermentation broth, fermentation product, or composition are the desired Methylobacterium or other desired microorganisms of pre-determined identity.

As used herein, the phrase “inanimate solid substance” refers to a substance which is insoluble or partially soluble in water or aqueous solutions and which is either non-living or which is not a part of a still-living organism from which it was derived.

As used herein, the phrase “mono-culture ofMethylobacterium” refers to a Methylobacterium culture consisting of a single strain of Methylobacterium.

As used herein, a “pesticide” refers to an insecticide, fungicide, nematocide, bacteriocide, or any combination thereof.

As used herein, the phrase “bacteriostatic agent” refers to agents that inhibit growth of bacteria but do not kill the bacteria.

As used herein, the phrase “pesticide does not substantially inhibit growth of said Methylobacterium” refers to any pesticide that when provided in a composition comprising a fermentation product comprising a solid substance wherein a mono-culture or co-culture of Methylobacterium is adhered thereto, results in no more than a 50% inhibition of Methylobacterium growth when the composition is applied to a plant or plant part in comparison to a composition lacking the pesticide. In certain embodiments, the pesticide results in no more than a 40%, 20%, 10%, 5%, or 1% inhibition of Methylobacterium growth when the composition is applied to a plant or plant part in comparison to a composition lacking the pesticide.

As used herein, the term “Methylobacterium” refers to genera and species in the methylobacteriaceae family, including bacterial species in the Methylobacterium genus and proposed Methylorubrum genus (Green and Ardley (2018)). Methylobacterium includes pink-pigmented facultative methylotrophic bacteria (PPFM) and also encompasses the non-pink-pigmented Methylobacterium nodulans, as well as colorless mutants of Methylobacterium isolates. For example, and not by way of limitation, “Methylobacterium” refers to bacteria of the species listed below as well as any new Methylobacterium species that have not yet been reported or described that can be characterized as Methylobacterium or Methylorubrum based on phylogenetic analysis: Methylobacterium adhaesivum; Methylobacterium oryzae; Methylobacterium aerolatum; Methylobacterium oxalidis; Methylobacterium aquaticum; Methylobacterium persicinum; Methylobacterium brachiatum; Methylobacterium phyllosphaerae; Methylobacterium brachythecii; Methylobacterium phyllostachyos; Methylobacterium bullatum; Methylobacterium platani; Methylobacterium cerastii; Methylobacterium pseudosasicola; Methylobacterium currus; Methylobacterium radiotolerans; Methylobacterium dankookense; Methylobacterium soli; Methylobacterium frigidaeris; Methylobacterium specialis; Methylobacterium fujisawaense; Methylobacterium tardum; Methylobacterium gnaphalii; Methylobacterium tarhaniae; Methylobacterium goesingense; Methylobacterium thuringiense; Methylobacterium gossipiicola; Methylobacterium trifolii; Methylobacterium gregans; Methylobacterium variabile; Methylobacterium haplocladii; Methylobacterium aminovorans (Methylorubrum aminovorans); Methylobacterium hispanicum; Methylobacterium extorquens (Methylorubrum extorquens); Methylobacterium indicum; Methylobacterium podarium (Methylorubrum podarium); Methylobacterium iners; Methylobacterium populi (Methylorubrum populi); Methylobacterium isbiliense; Methylobacterium pseudosasae (Methylorubrum pseudosasae); Methylobacterium jeotgali; Methylobacterium rhodesianum (Methylorubrum rhodesianum); Methylobacterium komagatae; Methylobacterium rhodinum (Methylorubrum rhodinum); Methylobacterium longum; Methylobacterium salsuginis (Methylorubrum salsuginis); Methylobacterium marchantiae; Methylobacterium suomiense (Methylorubrum suomiense; Methylobacterium mesophilicum; Methylobacterium thiocyanatum (Methylorubrum thiocyanatum); Methylobacterium nodulans; Methylobacterium zatmanii (Methylorubrum zatmanii); Methylobacterium organophilum .

As used herein, the phrase “solid substance” refers to a substance which is insoluble or partially soluble in water or aqueous solutions.

As used herein, the phrase “solid phase that can be suspended therein” refers to a solid substance that can be distributed throughout a liquid by agitation.

As used herein, the term “non-regenerable” refers to either a plant part or processed plant product that cannot be regenerated into a whole plant.

As used herein, the phrase “substantially all of the solid phase is suspended in the liquid phase” refers to media wherein at least 95%, 98%, or 99% of solid substance(s) comprising the solid phase are distributed throughout the liquid by agitation.

As used herein, the phrase “substantially all of the solid phase is not suspended in the liquid phase” refers to media where less than 5%, 2%, or 1% of the solid is in a particulate form that is distributed throughout the media by agitation.

To the extent to which any of the preceding definitions is inconsistent with definitions provided in any patent or non-patent reference incorporated herein by reference, any patent or non-patent reference cited herein, or in any patent or non-patent reference found elsewhere, it is understood that the preceding definition will be used herein.

### Yield Enhancing Methylobacterium, Compositions Comprising Yield Enhancing Methylobacterium, Methods of their Use, and Methods of Making

Various yield enhancing Methylobacterium isolates, compositions comprising these Methylobacterium, methods of using the compositions to improve corn plant yield, and methods of making the compositions are provided herein. In certain embodiments, yield enhancing Methylobacterium isolates include NLS0807 (NRRL B-67743), NLS0662 (NRRL B-67742), NLS0648 (NRRL B-67741), NLS0109 (NRRL B-67340) or variants thereof, or a combination of Methylobacterium isolate NLS0109 (NRRL B-67340) or a variant thereof and Methylobacterium isolate NLS0017 (B-50931) or a variant thereof. Amounts of the compositions that comprise yield enhancing Methylobacterium sufficient to provide for improved corn plant yield can be determined by measuring any or all of changes in yield relative to untreated plants or plant parts. In certain embodiments, yield can be assessed by measuring output of seed on a per unit area basis (i.e. bushels per acre, kilograms per hectare, and the like), where the yield enhancing Methylobacterium treated plants or plants grown from Methylobacterium treated seed are grown at about the same density as the control plants. In certain embodiments, yield can be assessed by measuring output on a per plant or per plant part basis (grams of seed per plant, grams of seed per cob, kernels per plant, kernels per cob and the like) of the yield enhancing Methylobacterium treated plants in comparison to untreated control plants.

Isolated yield enhancing Methylobacterium are provided herein. In certain embodiments, the Methylobacterium is selected from the group consisting of M. radiotolerans, M. komagatae, and M. gregans. In certain embodiments, the yield enhancing Methylobacterium isolate is selected from the group consisting of NLS0807 (NRRL B-67743), NLS0662 (NRRL B-67742), NLS0648 (NRRL B-67741), NLS0109 (NRRL B-67340), and variants thereof. In certain embodiments, the yield enhancing Methylobacterium is a combination of Methylobacterium isolates NLS0109 and NLS0017 (NRRL B-50931). In certain embodiments, the yield enhancingMethylobacterium isolate can enhance yield when applied to a corn seed, when applied in vegetative stages of corn development, or when applied during reproductive stages of corn development. In certain embodiments, the yield enhancing Methylobacterium has a chromosomal genomic DNA with at least 99%, 99.9, 99.8, 99.7, 99.6%, or 99.5% sequence identity to chromosomal genomic DNA of NLS0807 (NRRL B-67743), NLS0662 (NRRL B-67742), NLS0648 (NRRL B-67741), or NLS0109 (NRRL B-67340); or the yield enhancing Methylobacterium is a combination of Methylobacterium variants of NLS0109 and NLS0017 having chromosomal genomic DNA with at least 99%, 99.9, 99.8, 99.7, 99.6%, or 99.5% sequence identity to chromosomal genomic DNA of NLS0109 (NRRL B-67340) and NLS0017. In certain embodiments, the yield enhancing Methylobacterium provides for at least about 2%, at least about 3%, at least about 4%, at least about 5%, at least about 6%, at least about 7%, at least about 8%, at least about 9%, at least about 10%, or at least about 15% increases in yield of a treated plant or a plant arising from a treated seed in comparison to untreated control plants or plants grown from untreated seeds. In certain embodiments, the yield enhancing Methylobacterium provides for at least about 2%, at least about 3%, at least about 4%, at least about 5%, at least about 6%, at least about 7%, at least about 8%, at least about 9%, to at least about a 10% or at least about a 20% increase in yield of a treated plant or a plant grown from a treated seed in comparison to untreated control plants or plants arising from untreated seeds.

In certain embodiments, the yield enhancing Methylobacterium provides for increased yield when applied to a seed. In certain embodiments, the yield enhancing Methylobacterium provides for increases in yield when applied to seed in furrow at planting. In certain embodiments, the yield enhancing Methylobacterium provides for increases in yield when applied during corn vegetative stages of development. In certain embodiments, the yield enhancing Methylobacterium provides for increases in yield when applied just prior to or during corn reproductive stages of development. In certain embodiments of any of the aforementioned compositions, the composition comprises a solid substance wherein a mono-culture or co-culture of Methylobacterium is adhered thereto. In certain embodiments where the Methylobacterium is adhered to a solid substance, the composition comprises a colloid formed by the solid substance wherein a mono-culture or co-culture of Methylobacterium is adhered thereto and a liquid. In certain embodiments, the colloid is a gel. In certain embodiments of certain aforementioned compositions, composition is an emulsion that does not contain a solid substance. In certain embodiments of any of the aforementioned compositions, the yield enhancing Methylobacterium is selected from the group consisting of NLS0807 (NRRL B-67743), NLS0662 (NRRL B-67742), and variants thereof. In certain embodiments of any of the aforementioned compositions, the yield enhancingMethylobacterium is selected from the group consisting of NLS0648 (NRRL B-67741), NLS0109 (NRRL B-67340), and variants thereof. In certain embodiments of any of the aforementioned compositions, the yield enhancing Methylobacterium is a combination of Methylobacterium NLS0109 and NLS0017. In certain embodiments, the yield enhancing Methylobacterium has a chromosomal genomic DNA with at least 99%, 99.9, 99.8, 99.7, 99.6%, or 99.5% sequence identity to chromosomal genomic DNA of NLS0807 (NRRL B-67743), NLS0662 (NRRL B-67742), NLS0648 (NRRL B-67741), or NLS0109 (NRRL B-67340); or the yield enhancing Methylobacterium is a combination of Methylobacterium variants of NLS0109 and NLS0017 having chromosomal genomic DNA with at least 99%, 99.9, 99.8, 99.7, 99.6%, or 99.5% sequence identity to chromosomal genomic DNA of NLS0109 (NRRL B-67340) and NLS0017 (NRRL B-50931).

In certain embodiments, isolated yield enhancing Methylobacterium can be identified by treating a plant, a seed, soil in which the plant or a plant arising from the seed are grown, or other plant growth media in which the plant or a plant arising from the seed are grown and assaying for increased yield.

In certain embodiments, corn seed or corn plants in the vegetative stages of development are treated with the yield enhancing Methylobacterium The vegetative stages of corn are as follows: VE (coleoptile emergence to just prior to first leaf collaring), V1 (first leaf collared), V2 (first and second leaf collared), V3 (first through third leaf collared), V4 (first through fourth leaf collared), V5 (first through fifth leaf collared), V6 (first through sixth leaf collared), and so on up to V18 (plants with the 18^{th} leaf collared). A description of the corn vegetative stages can be found on the world wide web (internet) at agronext.iastate.edu/corn/production/management/growth/CornGrowthandDevelopment.html and in “Corn Growth and Development”, Abendroth et al. Iowa State University Extension and Outreach publication PMR 1009 March 2011). In certain embodiments, the yield enhancing Methylobacterium are applied at about the VE to about the V4, V5, or V6 stage of development. In certain embodiments, the yield enhancing Methylobacterium are applied at about the VE, V1, V2, or V3 to about the V4, V5, or V6 stage of development. In certain embodiments, the yield enhancing Methylobacterium that is applied to the seed or during a vegetative stage is NLS0807 (NRRL B-67743.

In certain embodiments, the yield enhancing Methylobacterium are applied at about the V5, V6 to about the Vn stage or VT stage of development to about the R2, R3, R4, R5, or R6 stage of development. In certain embodiments, the yield enhancing Methylobacterium are applied at about the V12, V16, V18, Vn, or VT stage of development to about the R2, R3, or R4 stage of development. In certain embodiments, the yield enhancing Methylobacterium are applied at about the R1 stage of development.

In certain embodiments, the yield enhancing Methylobacterium are applied to a transgenic corn plant that is herbicide, insect or disease tolerant. In certain embodiments, the yield enhancing Methylobacterium are applied before, during, or after the application of glyphosate to a transgenic corn plant that is glyphosate tolerant. Commercially available glyphosate formulations that can be used include, but are not limited to, Roundup Original MAX®, Roundup PowerMAX®, Roundup UltraMax®, or RoundUp WeatherMAX® (Monsanto Co., St. Louis, MO., USA); Touchdown IQ® or Touchdown Total® (Syngenta, Wilmington, Delaware, USA); Glyphomax®, Glyphomax Plus®, or Glyphomax XRT® (Dow Agrosciences LLC, Indianapolis, IN, USA). Corn plants at are typically sprayed with glyphosate at about the V3 and/or at about the V6 vegetative development stage. In certain embodiments, the yield enhancing Methylobacterium that is applied before, during, or after the application of glyphosate a Methylobacterium that is selected for glyphosate resistance. Selections for glyphosate resistant bacteria that have been described (Comai et al. (1983)) can be adapted for selection of yield enhancing Methylobacterium. The selection and use of glyphosate resistant yield enhancing Methylobacterium from mutagenized or other populations of Methylobacterium such as NLS0807 (NRRL B-67743), NLS0662 (NRRL B-67742), NLS0648 (NRRL B-67741), NLS0109 (NRRL B-67340), and variants thereof is provided herein.

Various Methylobacterium isolates provided herein are disclosed in Table 1.

TABLE 1
Methylobacterium isolates

| | ISOLATE | NLS | USDA ARS |
|---|---|---|---|
| Deposit Identifier | No. | No. | NRRL No.¹ |
| Methylobacterium sp. #1 | ISO01 | NLS0046 | NRRL B-50929 |
| Methylobacterium sp. #2 | ISO02 | NLS0020 | NRRL B-50930 |
| Methylobacterium sp. #3 | ISO03 | NLS0017 | NRRL B-50931 |
| Methylobacterium sp. #4 | ISO04 | NLS0042 | NRRL B-50932 |
| Methylobacterium sp. #5 | ISO05 | NLS0089 | NRRL B-50933 |
| Methylobacterium sp. #6 | ISO06 | NLS0068 | NRRL B-50934 |
| Methylobacterium sp. #7 | ISO07 | NLS0065 | NRRL B-50935 |
| Methylobacterium sp. #8 | ISO08 | NLS0069 | NRRL B-50936 |
| Methylobacterium sp. #9 | ISO09 | NLS0062 | NRRL B-50937 |
| Methylobacterium sp. #10 | ISO10 | NLS0064 | NRRL B-50938 |
| Methylobacterium sp. #11 | ISO11 | NLS0021 | NRRL B-50939 |
| Methylobacterium sp. #12 | ISO12 | NLS0066 | NRRL B-50940 |
| Methylobacterium sp. #13 | ISO13 | NLS0037 | NRRL B-50941 |
| Methylobacterium sp. #14 | ISO14 | NLS0038 | NRRL B-50942 |
| Methylobacterium #15 | ISO15 | NLS0044 | NRRL B-67339 |
| Methylobacterium #16 | ISO16 | NLS0109 | NRRL B-67340 |
| Methylobacterium sp (#18) | ISO18 | NLS0648 | NRRL B-67741 |
| Methylobacterium sp (#19) | ISO19 | NLS0662 | NRRL B-67742 |
| Methylobacterium sp (#20) | ISO20 | NLS0807 | NRRL B-67743 |

¹Deposit number for strain deposited with the AGRICULTURAL RESEARCH SERVICE CULTURE COLLECTION (NRRL) of the National Center for Agricultural Utilization Research, Agricultural Research Service, U.S. Department of Agriculture, 1815 North University Street, Peoria, Illinois 61604 U.S.A. under the terms of the Budapest Treaty on the International Recognition of the Deposit of Microorganisms for the Purposes of Patent Procedure. Subject to 37 CFR §1.808(b), all restrictions imposed by the depositor on the availability to the public of the deposited material will be irrevocably removed upon the granting of any patent from this patent application.

Samples of the following Methylobacterium sp. strains have been deposited with the AGRICULTURAL RESEARCH SERVICE CULTURE COLLECTION (NRRL) of the National Center for Agricultural Utilization Research, Agricultural Research Service, U.S. Department of Agriculture, 1815 North University Street, Peoria, Illinois 61604 U.S.A. under the terms of the Budapest Treaty on the International Recognition of the Deposit of Microorganisms for the Purposes of Patent Procedure. Methylobacterium sp. NRRL B-50929, NRRL B-50930, NRRL B-50931, NRRL B-50932, NRRL B-50933, NRRL B-50934, NRRL B-50935, NRRL B-50936, NRRL B-50937, NRRL B-50938, NRRL B-50939, NRRL B-50940, NRRL B-50941 and NRRL B-50942 were deposited with NRRL on Mar. 12, 2014. Methylobacterium sp. NRRL B-67339, NRRL B-67340, and NRRL B-67341 were deposited with NRRL on Nov. 18, 2016. Methylobacterium sp. NRRL B-67741, NRRL B-67742, and NRRL B-67743 were deposited with NRRL on Dec. 20, 2018.

Variants of a Methylobacterium isolate listed in Table 1 include isolates obtained therefrom by genetic transformation, mutagenesis and/or insertion of a heterologous sequence. In some embodiments, such variants are identified by the presence of chromosomal genomic DNA with at least 99%, 99.9, 99.8, 99.7, 99.6%, or 99.5% sequence identity to chromosomal genomic DNA of the strain from which it was derived. In certain embodiments, such variants are distinguished by the presence of one or more unique DNA sequences that include: (i) a unique sequence of SEQ ID NOs: 1 to 3, SEQ ID NOs: 13 to 15, SEQ ID NOs: 25-27, SEQ ID NOs: 37-39, SEQ ID NOs: 49 to 51, and SEQ ID NOs: 61-73; or (ii) sequences with at least 98% or 99% sequence identity across the full length of SEQ ID NOs: 1 to 3, SEQ ID NOs: 13 to 15, SEQ ID NOs: 25-27, SEQ ID NOs: 37-39, SEQ ID NOs: 49 to 51, and SEQ ID NOs: 61-73.

Co-assigned patent applications that disclose additional specific uses of certain Methylobacterium strains of Table 1 such as: increasing soybean yield (U.S. Patent Appl. Pub. No. 20160302423); improving lettuce cultivation (U.S. Pat. No. 10,212,939); improving tomato growth (U.S. Patent Appl. Pub. No. 20170086464); improving corn yield (U.S. Patent Appl. Pub. No. 20160295868); improving soy yield (US2016/0302423); improving fruit production (U.S. Pat. No. 10,111,438); controlling corn rootworm (US20170238553); controlling root lesion nematodes (US20170164618); controlling root knot nematodes (US20170135352); and controlling fungal disease (US20180295841 and WO2018106899) and are each incorporated herein by reference.

Also provided herein are methods for improving corn yield that comprise applying any of the aforementioned compositions provided herein to a plant or a plant part in an amount that provides for increased corn yield in the plant, plant part, or a plant obtained therefrom relative to yield of a control plant, plant part, or plant obtained therefrom that had not received an application of the composition. In certain embodiments, the plant part is selected from the group consisting of a leaf, a stem, a flower, a root, a cob, and a seed. In certain embodiments, the method further comprises the step of harvesting at least one plant part selected from the group consisting of a leaf, a stem, a flower, a root, a cob, or a seed from the plant or plant part. In certain embodiments of any of the aforementioned methods, the methods further comprise obtaining a processed food or feed composition from the plant or plant part. In certain embodiments, the processed food or feed composition is a meal or a paste. In certain embodiments of any of the aforementioned methods, the yield enhancing Methylobacterium is selected from the group consisting of NLS0807 (NRRL B-67743), NLS0662 (NRRL B-67742), NLS0648 (NRRL B-67741), or NLS0109 (NRRL B-67340), and variants thereof. In certain embodiments of any of the aforementioned compositions, the yield enhancing Methylobacterium is a combination of Methylobacterium NLS0109 and NLS0017, or variants thereof.

Also provided are methods of making the compositions useful for improving corn yield that comprise combining a yield enhancing Methylobacterium with an agriculturally acceptable excipient and/or with an agriculturally acceptable adjuvant. In certain embodiments of the methods, the Methylobacterium is adhered to a solid substance. In certain embodiments of the methods, the Methylobacterium is adhered to the solid substance is combined with a liquid to form a composition that is a colloid. In certain embodiments of the methods, the colloid is a gel. In certain embodiments of the methods, the Methylobacterium adhered to the solid substance is provided by culturing the Methylobacterium in the presence of the solid substance. In certain embodiments of the methods, the composition comprises an emulsion. In certain embodiments of the methods, the Methylobacterium is provided by culturing the Methylobacterium in an emulsion. In certain embodiments of any of the aforementioned methods, the yield enhancing Methylobacterium is selected from the group consisting of NLS0807 (NRRL B-67743), NLS0662 (NRRL B-67742), and variants thereof. In certain embodiments of any of the aforementioned methods, the yield enhancing Methylobacterium is selected from the group consisting of NLS0648 (NRRL B-67741), NLS0109 (NRRL B-67340), and variants thereof. In certain embodiments of any of the aforementioned compositions, the yield enhancing Methylobacterium is a combination of Methylobacterium NLS0109 and NLS0017, or variants thereof.

Methods where Methylobacterium are cultured in biphasic media comprising a liquid phase and a solid substance have been found to significantly increase the resultant yield of Methylobacterium relative to methods where the Methylobacterium are cultured in liquid media alone. In certain embodiments, the methods can comprise growing the Methylobacterium in liquid media with a particulate solid substance that can be suspended in the liquid by agitation under conditions that provide for Methylobacterium growth. In certain embodiments where particulate solid substances are used, at least substantially all of the solid phase can thus be suspended in the liquid phase upon agitation. Such particulate solid substances can comprise materials that are about 1 millimeter or less in length or diameter. In certain embodiments, the degree of agitation is sufficient to provide for uniform distribution of the particulate solid substance in the liquid phase and/or optimal levels of culture aeration. However, in other embodiments provided herein, at least substantially all of the solid phase is not suspended in the liquid phase, or portions of the solid phase are suspended in the liquid phase and portions of the solid phase are not suspended in the liquid phase. Non-particulate solid substances can be used in certain biphasic media where the solid phase is not suspended in the liquid phase. Such non-particulate solid substances include, but are not limited to, materials that are greater than about 1 millimeter in length or diameter. Such particulate and non-particulate solid substances also include, but are not limited to, materials that are porous, fibrous, or otherwise configured to provide for increased surface areas for adherent growth of the Methylobacterium. Biphasic media where portions of the solid phase are suspended in the liquid phase and portions of the solid phase are not suspended in the liquid phase can comprise a mixture of particulate and non-particulate solid substances. Such particulate and non-particulate solid substances used in any of the aforementioned biphasic media also include, but are not limited to, materials that are porous, fibrous, or otherwise configured to provide for increased surface areas for adherent growth of the Methylobacterium. In certain embodiments, the media comprises a colloid formed by a solid and a liquid phase. A colloid comprising a solid and a liquid can be pre-formed and added to liquid media or can be formed in media containing a solid and a liquid. Colloids comprising a solid and a liquid can be formed by subjecting certain solid substances to a chemical and/or thermal change. In certain embodiments, the colloid is a gel. In certain embodiments, the liquid phase of the media is an emulsion. In certain embodiments, the emulsion comprises an aqueous liquid and a liquid that is not miscible, or only partially miscible, in the aqueous liquid. Liquids that are not miscible, or only partially miscible, in water include, but are not limited to, any of the following: (1) liquids having a miscibility in water that is equal to or less than that of pentanol, hexanol, or heptanol at 25 degrees C.; (2) liquids comprising an alcohol, an aldehyde, a ketone, a fatty acid, a phospholipid, or any combination thereof; (3) alcohols selected from the group consisting of aliphatic alcohols containing at least 5 carbons and sterols; (4) an animal oil, microbial oil, synthetic oil, plant oil, or combination thereof; and/or, (5) a plant oil is selected from the group consisting of corn, soybean, cotton, peanut, sunflower, olive, flax, coconut, palm, rapeseed, sesame seed, safflower, and combinations thereof. In certain embodiments, the immiscible or partially immiscible liquid can comprise at least about 0.02% to about 20% of the liquid phase by mass. In certain embodiments, the methods can comprise obtaining a biphasic culture media comprising the liquid, the solid, and Methylobacterium and incubating the culture under conditions that provide for growth of the Methylobacterium. Biphasic culture medias comprising the liquid, the solid, and Methylobacterium can be obtained by a variety of methods that include, but are not limited to, any of: (a) inoculating a biphasic media comprising the liquid and the solid substance with Methylobacterium; (b) inoculating the solid substance with Methylobacterium and then introducing the solid substance comprising the Methylobacterium into the liquid media; (c) inoculating the solid substance with Methylobacterium, incubating the Methylobacterium on the solid substance, and then introducing the solid substance comprising the Methylobacterium into the liquid media; or (d) any combination of (a), (b), or (c). Methods and compositions for growing Methylobacterium in biphasic media comprising a liquid and a solid are disclosed in co-assigned U.S. Pat. No. 9,181,541 and 9,845,462, which are incorporated herein by reference in their entirety, and in co-assigned International Patent Publication WO2013181610, published Dec. 5, 2013, which is incorporated herein by reference in its entirety.

Methods where Methylobacterium are cultured in media comprising an emulsion have also been found to significantly increase the resultant yield of Methylobacterium relative to methods where the Methylobacterium are cultured in liquid media alone. In certain embodiments, the methods for making the compositions provided herein can comprise growing the yield enhancing Methylobacterium in an emulsion under conditions that provide for Methylobacterium growth. Medias comprising the emulsion and yield enhancing Methylobacterium can be obtained by a variety of methods that include, but are not limited to, any of: (a) inoculating a media comprising the emulsion with Methylobacterium; (b) inoculating the aqueous liquid with the Methylobacterium, introducing the non-aqueous liquid, and mixing to form an emulsion; (c) inoculating the aqueous liquid with the Methylobacterium, introducing the non-aqueous liquid, and mixing to form an emulsion; or (d) any combination of (a), (b), or (c). In certain embodiments, the emulsion comprises an aqueous liquid and a liquid that is not miscible, or only partially miscible, in the aqueous liquid. Non-aqueous liquids that are not miscible, or only partially miscible, in water include, but are not limited to, any of the following: (1) liquids having a miscibility in water that is equal to or less than that of n-pentanol, n-hexanol, or n-heptanol at 25 degrees C.; (2) liquids comprising an alcohol, an aldehyde, a ketone, a fatty acid, a phospholipid, or any combination thereof; (3) alcohols is selected from the group consisting of aliphatic alcohols containing at least 5, 6, or 7 carbons and sterols; (4) an animal oil, microbial oil, synthetic oil, plant oil, or combination thereof; and/or, (5) a plant oil is selected from the group consisting of corn, soybean, cotton, peanut, sunflower, olive, flax, coconut, palm, rapeseed, sesame seed, safflower, and combinations thereof. In certain embodiments, the immiscible or partially immiscible non-aqueous liquid can comprise at least about 0.02% to about 20% of the emulsion by mass. In certain embodiments, the immiscible or partially immiscible non-aqueous liquid can comprise at least about any of about 0.05%, 0.1%, 0.5%, or 1% to about 3%, 5%, 10%, or 20% of the emulsion by mass. Methods and compositions for growing Methylobacterium in media comprising an emulsion are disclosed in co-assigned U.S. Pat. No. 10,287,544 and International Patent Publication WO2014194189, published Dec. 4, 2014, which is incorporated herein by reference in its entirety.

In some embodiments, the composition or method disclosed herein may comprise one or more additional components. In some embodiments a second component can be an additional active ingredient, for example, a pesticide or a second biological. The pesticide may be, for example, an insecticide, a fungicide, an herbicide, or a nematicide. The second biological can be a biocontrol agent.

Non-limiting examples of insecticides and nematicides include carbamates, diamides, macrocyclic lactones, neonicotinoids, organophosphates, phenylpyrazoles, pyrethrins, spinosyns, synthetic pyrethroids, tetronic and tetramic acids. In particular embodiments insecticides and nematicides include abamectin, aldicarb, aldoxycarb, bifenthrin, carbofuran, chlorantraniliporle, chlothianidin, cyfluthrin, cyhalothrin, cypermethrin, deltamethrin, dinotefuran, emamectin, ethiprole, fenamiphos, fipronil, flubendiamide, fosthiazate, imidacloprid, ivermectin, lambda-cyhalothrin, milbemectin, nitenpyram, oxamyl, permethrin, tioxazafen, spinetoram, spinosad, spirodichlofen, spirotetramat, tefluthrin, thiacloprid, thiamethoxam, and thiodicarb.

Non-limiting examples of useful fungicides include aromatic hydrocarbons, benzimidazoles, benzthiadiazole, carboxamides, carboxylic acid amides, morpholines, phenylamides, phosphonates, quinone outside inhibitors (e.g. strobilurins), thiazolidines, thiophanates, thiophene carboxamides, and triazoles. Particular examples of fungicides include acibenzolar-S-methyl, azoxystrobin, benalaxyl, bixafen, boscalid, carbendazim, cyproconazole, dimethomorph, epoxiconazole, fluopyram, fluoxastrobin, flutianil, flutolanil, fluxapyroxad, fosetyl-Al, ipconazole, isopyrazam, kresoxim-methyl, mefenoxam, metalaxyl, metconazole, myclobutanil, orysastrobin, penflufen, penthiopyrad, picoxystrobin, propiconazole, prothioconazole, pyraclostrobin, sedaxane, silthiofam, tebuconazole, thifluzamide, thiophanate, tolclofos-methyl, trifloxystrobin, and triticonazole.

Non-limiting examples of herbicides include ACCase inhibitors, acetanilides, AHAS inhibitors, carotenoid biosynthesis inhibitors, EPSPS inhibitors, glutamine synthetase inhibitors, PPO inhibitors, PS II inhibitors, and synthetic auxins, Particular examples of herbicides include acetochlor, clethodim, dicamba, flumioxazin, fomesafen, glyphosate, glufosinate, mesotrione, quizalofop, saflufenacil, sulcotrione, and 2,4-D.

In some embodiments, compositions or methods disclosed herein may comprise an additional active ingredient which may be a second biological. The second biological could be a biocontrol agent, other beneficial microorganisms, microbial extracts, natural products, plant growth activators or a plant defense agent. Non-limiting examples of biocontrol agents include bacteria, fungi, beneficial nematodes, and viruses.

In certain embodiments, the second biological can be Methylobacterium. In certain embodiments, the second biological is a Methylobacterium listed in Table 1. In certain embodiments, the second biological is selected from the group consisting of ISO01 (NRRL B-50929), ISO02 (NRRL B-50930), ISO03 (NRRL B-50931), ISO04 (NRRL B-50932), ISO05 (NRRL B-50933), ISO06 (NRRL B-50934), ISO07 (NRRL B-50935), ISO08 (NRRL B-50936), ISO09 (NRRL B-50937), ISO10 (NRRL B-50938), ISO11 (NRRL B-50939), ISO12 (NRRL B- 50940), ISO13 (NRRL B-50941), and ISO14 (NRRL B-50942). In certain embodiments, the second biological can be a Methylobacterium having chromosomal genomic DNA with at least 99%, 99.9, 99.8, 99.7, 99.6%, or 99.5% sequence identity to chromosomal genomic DNA of ISO01 (NRRL B-50929), ISO02 (NRRL B-50930), ISO03 (NRRL B-50931), ISO04 (NRRL B-50932), ISO05 (NRRL B-50933), ISO06 (NRRL B-50934), ISO07 (NRRL B-50935), ISO08 (NRRL B-50936), ISO09 (NRRL B-50937), ISO10 (NRRL B-50938), ISO11 (NRRL B-50939), ISO12 (NRRL B-50940), ISO13 (NRRL B-50941), or ISO14 (NRRL B-50942). In certain embodiments, the second biological can be a Methylobacterium selected from M. gregans, M. radiotolerans, M. extorquens, M. populi, M. salsuginis, M. brachiatum, and M. komagatae.

In certain embodiments, the second biological can be a bacterium of the genus Actinomycetes, Agrobacterium, Arthrobacter, Alcaligenes, Aureobacterium, Azobacter, Beijerinckia, Brevibacillus, Burkholderia, Chromobacterium, Clostridium, Clavibacter, Comomonas, Corynebacterium, Curtobacterium, Enterobacter, Flavobacterium, Gluconobacter, Hydrogenophage, Klebsiella, Methylobacterium, Paenibacillus, Pasteuria, Phingobacterium, Photorhabdus, Phyllobacterium, Pseudomonas, Rhizobium, Bradyrhizobium, Serratia, Stenotrophomonas, Variovorax, and Xenorhadbus. In particular embodiments the bacteria is selected from the group consisting of Bacillus amyloliquefaciens, Bacillus cereus, Bacillus firmus, Bacillus, lichenformis, Bacillus pumilus, Bacillus sphaericus, Bacillus subtilis, Bacillus thuringiensis, Chromobacterium suttsuga, Pasteuria penetrans, Pasteuria usage, and Pseudomona fluorescens.

In certain embodiments the second biological can be a fungus of the genus Alternaria, Ampelomyces, Aspergillus, Aureobasidium, Beauveria, Colletotrichum, Coniothyrium, Gliocladium, Metarhisium, Muscodor, Paecilonyces, Trichoderma, Typhula, Ulocladium, and Verticilium. In particular embodiments the fungus is Beauveria bassiana, Coniothyrium minitans, Gliocladium vixens, Muscodor albus, Paecilomyces lilacinus, or Trichoderma polysporum.

In further embodiments the second biological can be a plant growth activator or plant defense agent including, but not limited to harpin, Reynoutria sachalinensis, jasmonate, lipochitooligosaccharides, and isoflavones.

In further embodiments, the second biological can include, but is not limited to, various Bacillus sp., Pseudomonas sp., Coniothyrium sp., Pantoea sp., Streptomyces sp., and Trichoderma sp. Microbial biopesticides can be a bacterium, fungus, virus, or protozoan. Particularly useful biopesticidal microorganisms include various Bacillus subtilis, Bacillus thuringiensis, Bacillus pumilis, Pseudomonas syringae, Trichoderma harzianum, Trichoderma virens, and Streptomyces lydicus strains. Other microorganisms that are added can be genetically engineered or wild-type isolates that are available as pure cultures. In certain embodiments, it is anticipated that the biological or biocontrol agent can be provided in the fermentation broth, fermentation broth product, or composition in the form of a spore.

In certain embodiments, the fermentation broth, fermentation broth product, or compositions that comprise yield enhancing Methylobacterium can further comprise one or more introduced additional active ingredient or microorganism of pre-determined identity other than Methylobacterium.

In certain embodiments, the liquid culture medium is prepared from inexpensive and readily available components, including, but not limited to, inorganic salts such as potassium phosphate, magnesium sulfate and the like, carbon sources such as glycerol, methanol, glutamic acid, aspartic acid, succinic acid and the like, and amino acid blends such as peptone, tryptone, and the like. Exemplary liquid media that can be used include, but are not limited to, ammonium mineral salts (AMS) medium (Whittenbury et al., 1970), Vogel-Bonner (VB) minimal culture medium (Vogel and Bonner, 1956), and LB broth (“Luria-Bertani Broth”).

In general, a solid substance that can be used in the methods and compositions to provide for the efficient growth of Methylobacterium can be any suitable solid substance which is insoluble or only partially soluble in water or aqueous solutions. Such suitable solid substances are also non-bacteriocidal or non-bacteriostatic with respect to yield enhancing Methylobacterium when the solid substances are provided in the liquid culture media. In certain embodiments, such suitable solid substances are also solid substances that are readily obtained in sterile form or rendered sterile. Solid substances can be sterilized by any method that provides for removal of contaminating microorganisms and thus include, but are not limited to, methods such as autoclaving, irradiation, chemical treatment, and any combination thereof. These solid substances include natural substances of animal, plant, microbial, fungal, or mineral origin, manmade substances, or combinations of natural and manmade substances. In certain embodiments, the solid substances are inanimate solid substances. Inanimate solid substances of animal, plant, microbial, or fungal origin can be obtained from animals, plants, microbes, or fungi that are inviable (i.e. no longer living) or that have been rendered inviable. Diatom shells are thus inanimate solid substances when previously associated diatom algae have been removed or otherwise rendered inviable. Since diatom shells are inanimate solid substances, they are not considered to be photosynthetic organisms or photosynthetic microorganisms. In certain embodiments, solid substances include, but are not limited to, sand, silt, soil, clay, ash, charcoal, diatomaceous earth and other similar minerals, ground glass or glass beads, ground ceramic materials, ceramic beads, bentonite, kaolin, talc, perlite, mica, vermiculite, silicas, quartz powder, montmorillonite, and combinations thereof. In certain embodiments, a solid substance can be a polymer or polymeric beads. Polymers that can be used as a solid substance include, but are not limited to, various polysaccharides such as cellulosic polymers and chitinous polymers which are insoluble or only partially soluble in water or aqueous solutions, agar (i.e. galactans), and combinations thereof. In certain embodiments, a solid substance can be an insoluble or only partially soluble salt crystal. Salt crystals that can be used include, but are not limited to, insoluble or only partially soluble carbonates, chromates, sulfites, phosphates, hydroxides, oxides, and sulfides. In certain embodiments, a solid substance can be a microbial cell, fungal cell, microbial spore, or fungal spore. In certain embodiments, a solid substance can be a microbial cell or microbial spore wherein the microbial cell or microbial spore is not a photosynthetic microorganism. In certain embodiments, the microbial cell or microbial spore is not a photosynthetic microorganism, where the photosynthetic microorganism is selected from the group consisting of algae, cyanobacteria, diatoms, Botryococcus braunii, Chlorella, Dunaliella tertiolecta, Gracilaria, Pleurochrysis carterae, Sargassum, and Ulva. In still other embodiments, a solid substance can be an inactivated (i.e. inviable) microbial cell, fungal cell, microbial spore, or fungal spore. In still other embodiments, a solid substance can be a quiescent (i.e. viable but not actively dividing) microbial cell, fungal cell, microbial spore, or fungal spore. In still other embodiments, a solid substance can be cellular debris of microbial origin. In still other embodiments, a solid substance can be particulate matter from any part of a plant. Plant parts that can be used to obtain the solid substance include, but are not limited to, cobs, husks, hulls, leaves, roots, flowers, stems, barks, seeds, and combinations thereof. Products obtained from processed plant parts including, but not limited to, bagasse, wheat bran, soy grits, crushed seed cake, stover, and the like can also be used. Such plant parts, processed plants, and/or processed plant parts can be milled to obtain the solid material in a particulate form that can be used. In certain embodiments, wood or a wood product including, but not limited to, wood pulp, sawdust, shavings, and the like can be used. In certain embodiments, the solid substance can be a particulate matter from an animal(s), including, but not limited to, bone meal, gelatin, ground or powdered shells, hair, macerated hide, and the like.

In certain embodiments, a solid substance is provided in a particulate form that provides for distribution of the solid substance in the culture media. In certain embodiments, a solid substance is comprised of particle of about 2 microns to about 1000 microns in average length or average diameter. In certain embodiments, a solid substance is comprised of particle of about 1 micron to about 1000 microns in average length or average diameter. In certain embodiments, a solid substance is a particle of about 1, 2, 4, 10, 20, or 40 microns to any of about 100, 200, 500, 750, or 1000 microns in average length or average diameter. Desirable characteristics of particles used in the methods and compositions provided herein include suitable wettability such that the particles can be suspended throughout the media upon agitation.

In certain embodiments, a solid substance is provided in the media as a colloid wherein the continuous phase is a liquid and the dispersed phase is the solid. Suitable solids that can be used to form colloids in liquid media used to grow yield enhancing Methylobacterium include, but are not limited to, various solids that are referred to as hydrocolloids. Such hydrocolloids used in the media, methods and compositions provided herein can be hydrophilic polymers, of plant, animal, microbial, or synthetic origin. Hydrocolloid polymers used in the methods can contain many hydroxyl groups and/or can be polyelectrolytes. Hydrocolloid polymers used in the compositions and methods provided herein include, but are not limited to, agar, alginate, arabinoxylan, carrageenan, carboxymethylcellulose, cellulose, curdlan, gelatin, gellan, β-glucan, guar gum, gum arabic, locust bean gum, pectin, starch, xanthan gum, and mixtures thereof. In certain embodiments, a colloid used in the media, methods, and compositions provided herein can comprise a hydrocolloid polymer and one or more proteins.

In certain embodiments, a solid substance can provide for adherent growth of the yield enhancing Methylobacterium on the solid substance. Yield enhancing Methylobacterium that are adhered to a solid substance are Methylobacterium that cannot be substantially removed by simply washing the solid substance with the adherent yield enhancing Methylobacterium with growth media whereas non-adherent Methylobacterium can be substantially removed by washing the solid substance with liquid growth media. In this context, “substantially removed” means that at least about 30%, 40%, 50%, 60%, 70%, or 80% the Methylobacterium present are removed when the solid substance is washed with three volumes of liquid growth media. Such washing can be effected by a variety of methods including, but not limited to, decanting liquid from a washed solid phase or passing liquid through a solid phase on a filter that permits flow through of bacteria in the liquid. In certain embodiments, the adherent yield enhancing Methylobacterium that are associated with the solid can include both Methylobacterium that are directly attached to a solid and/or Methylobacterium that are indirectly attached to a solid substance. Methylobacterium that are indirectly attached to a solid substance include, but are not limited to, Methylobacterium that are attached to another Methylobacterium or to another microorganism that is attached to a solid substance, Methylobacterium that are attached to a solid substance by being attached to another substance that is attached to a solid substance, and the like. In certain embodiments, at least 10%, 20%, 30%, 40%, 50%, 60%, 70%, 80%, 90%, 95%, 98%, 99%, 99.5% or 99.9% of the Methylobacterium in the fermentation broth, fermentation broth product, or compositions are Methylobacterium that are adhered to a solid substance. In certain embodiments, adherent yield enhancing Methylobacterium can be present on the surface of a solid substance in the fermentation broth, fermentation broth product, or composition at a density of at least about 1 Methylobacterium/20 square micrometers, of at least about 1 Methylobacterium/10 square micrometers, of at least about 1 Methylobacterium/10 square micrometers, of at least about 1 Methylobacterium/5 square micrometers, of at least about 1 Methylobacterium/2 square micrometers, or of at least about 1 Methylobacterium/square micrometer. In certain embodiments, adherent yield enhancing Methylobacterium can be present on the surface of a solid substance in the fermentation broth, fermentation broth product, or composition at a density of at least about 1 Methylobacterium/20 square micrometers to about 1 Methylobacterium/square micrometer, of at least about 1 Methylobacterium/10 square micrometers to about 1 Methylobacterium/square micrometer, of at least about 1 Methylobacterium/10 square micrometers to about 1 Methylobacterium/square micrometer, of at least about 1 Methylobacterium/5 square micrometers to about 1 Methylobacterium/square micrometer, or of at least about 1 Methylobacterium/2 square micrometers to about 1 Methylobacterium/square micrometer. In certain embodiments, adherent yield enhancing Methylobacterium can be present on the surface of a solid substance in the fermentation broth, fermentation broth product, or composition at a density of at least about 1 Methylobacterium/20 square micrometers to about 1 Methylobacterium/2 square micrometers, of at least about 1 Methylobacterium/10 square micrometers to about 1 Methylobacterium/2 square micrometers, of at least about 1 Methylobacterium/10 square micrometers to about 1 Methylobacterium/2 square micrometers, or of at least about 1 Methylobacterium/5 square micrometers to about 1 Methylobacterium/2 square micrometers. Biphasic fermentation broths provided herein can comprise a liquid phase that contains non-adherent Methylobacterium. In certain embodiments, titers of non-adherent Methylobacterium in the liquid phase can be less than about 100,000, 10,000, or 1,000 CFU/ml. In certain embodiments of any of the aforementioned compositions, the yield enhancing Methylobacterium is selected from the group consisting of NLS0807 (NRRL B-67743), NLS0662 (NRRL B-67742), and variants thereof. In certain embodiments of any of the aforementioned compositions, the yield enhancing Methylobacterium is selected from the group consisting of NLS0648 (NRRL B-67741), NLS0109 (NRRL B-67340), and variants thereof. In certain embodiments of any of the aforementioned compositions, the yield enhancing Methylobacterium is a combination of Methylobacterium NLS0109 and NLS0017, or variants thereof.

Fermentation broths with yield enhancing Methylobacterium at a titer of greater than about 5×10⁸ colony-forming units per milliliter, at a titer of greater than about 1×10⁹ colony-forming units per milliliter, at a titer of greater than about 1×10¹⁰ colony-forming units per milliliter, at a titer of at least about 3×10¹⁰ colony-forming units per milliliter are provided herein. In certain embodiments, fermentation broths provided herein can comprise yield enhancing Methylobacterium at a titer of at least about 5×10⁸ colony-forming units per milliliter to at least about 3×10¹⁰ colony-forming units per milliliter, at least about 5×10⁸ colony-forming units per milliliter to at least about 4×10¹⁰ colony-forming units per milliliter, or at least about 5×10⁸ colony-forming units per milliliter to at least about 6×10¹⁰ colony-forming units per milliliter. In certain embodiments, fermentation broths provided herein can comprise yield enhancing Methylobacterium at a titer of at least about 1×10⁹ colony-forming units per milliliter to at least about 3×10¹⁰ colony-forming units per milliliter, at least about 1×10⁹ colony-forming units per milliliter to at least about 4×10¹⁰ colony-forming units per milliliter, or at least about 1×10⁹ colony-forming units per milliliter to at least about 6×10¹⁰ colony-forming units per milliliter. In certain embodiments, fermentation broths provided herein will comprise yield enhancing Methylobacterium at a titer of at least about 1×10¹⁰ colony-forming units per milliliter to at least about 3×10¹⁰ colony-forming units per milliliter, at least about 1×10¹⁰ colony-forming units per milliliter to at least about 4×10¹⁰ colony-forming units per milliliter, or at least about 1×10¹⁰ colony-forming units per milliliter to at least about 6×10¹⁰ colony-forming units per milliliter. In certain embodiments, fermentation broths provided herein will comprise yield enhancing Methylobacterium at a titer of, at least about 3×10¹⁰ colony-forming units per milliliter to at least about 4×10¹⁰ colony-forming units per milliliter, or at least about 3×10¹⁰ colony-forming units per milliliter to at least about 6×10¹⁰ colony-forming units per milliliter. In certain embodiments of any of the aforementioned compositions, the yield enhancing Methylobacterium is selected from the group consisting of NLS0807 (NRRL B-67743), NLS0662 (NRRL B-67742), and variants thereof. In certain embodiments of any of the aforementioned compositions, the yield enhancing Methylobacterium is selected from the group consisting of NLS0648 (NRRL B-67741), NLS0109 (NRRL B-67340), and variants thereof. In certain embodiments of any of the aforementioned compositions, the yield enhancing Methylobacterium is a combination of Methylobacterium NLS0109 and NLS0017, or variants thereof.

Yield enhancing Methylobacterium can be obtained as fermentation products and used to make various compositions useful for treating plants or plant parts to improve corn yield. The Methylobacterium compositions can be applied to plants or plant parts in various forms, including for example as liquid compositions or as dried compositions, for example dried powders. Plants or plant parts that have been at least partially coated or coated with the fermentation broth products or compositions comprising yield enhancing Methylobacterium are thus provided. In certain embodiments, the plant part is a seed. Partial coating of a plant, or a plant part, such as a seed includes, but is not limited to coating at least about 5%, 10%, 20%, 30%, 40%, 50%, 60%, 70%, 80%, 90%, 95%, 98%, 99%, or about 99.5% of the surface area of the plant, plant part, or plant seed. Also provided are processed plant products that contain the fermentation broth products or compositions with yield enhancing Methylobacterium or adherent yield enhancing Methylobacterium. In some embodiments solid substances with adherent yield enhancing Methylobacterium can be used to make various compositions that are particularly useful for treating plant seeds. Seeds that have been at least partially coated with the fermentation broth products or compositions are thus provided. Partial coating of a seed includes, but is not limited to coating at least about 5%, 10%, 20%, 30%, 40%, 50%, 60%, 70%, 80%, 90%, 95%, 98%, 99%, or about 99.5% of the surface area of the seed. Also provided are processed seed products, including, but not limited to, meal, flour, feed, and flakes that contain the fermentation broth products or compositions provided herein. In certain embodiments, the processed plant product will be non-regenerable (i.e. will be incapable of developing into a plant). In certain embodiments, the solid substance used in the fermentation product or composition that at least partially coats the plant or plant part or that is contained in the processed plant or plant part product comprises a solid substance and associated or adherent yield enhancing Methylobacterium that can be readily identified by comparing a treated and an untreated plant or plant part or processed product thereof. In certain embodiments, the yield enhancing Methylobacterium is selected from the group consisting of NLS0807 (NRRL B-67743), NLS0662 (NRRL B-67742), and variants thereof. In certain embodiments, the yield enhancing Methylobacterium is selected from the group consisting of NLS0648 (NRRL B-67741), NLS0109 (NRRL B-67340), and variants thereof. In certain embodiments of any of the aforementioned compositions, the yield enhancing Methylobacterium is a combination of Methylobacterium NLS0109 and NLS0017, or variants thereof.

Compositions useful for treating plants or plant parts that comprise yield enhancing Methylobacterium can also further comprise additional components, including an active ingredient, an agriculturally acceptable adjuvant or an agriculturally acceptable excipient. An agriculturally acceptable adjuvant or an agriculturally acceptable excipient is typically an ingredient that does not cause undue phytotoxicity or other adverse effects when exposed to a plant or plant part. In certain embodiments, a solid substance used in a fermentation broth can itself be an agriculturally acceptable adjuvant or an agriculturally acceptable excipient so long as it is not bacteriocidal or bacteriostatic to the Methylobacterium. In some embodiments agriculturally acceptable adjuvants and/or excipients are added to Methylobacterium to increase stability and/or shelf life. In other embodiments, the composition further comprises at least one of an agriculturally acceptable adjuvant or an agriculturally acceptable excipient. Any of the aforementioned compositions can also further comprise an additional active ingredient. In certain embodiments, the additional active ingredient is a pesticide used in the composition that does not substantially inhibit growth of the Methylobacterium. As Methylobacterium are gram negative bacteria, suitable bacteriocides used in the compositions can include, but are not limited to, bacteriocides that exhibit activity against gram positive bacteria but not gram negative bacteria. Compositions provided herein can also comprise a bacteriostatic agent that does not substantially inhibit growth of the Methylobacterium. Bacteriostatic agents suitable for use in compositions provided herein include, but are not limited to, those that exhibit activity against gram positive bacteria but not gram negative bacteria. Any of the aforementioned compositions can also be an essentially dry product (i.e. having about 5% or less water content), a mixture of the composition with an emulsion, or a suspension.

Agriculturally acceptable adjuvants used in the compositions that comprise yield enhancing Methylobacterium include, but are not limited to, components that enhance product efficacy and/or products that enhance ease of product application. Adjuvants that enhance product efficacy can include various wetters/spreaders that promote adhesion to and spreading of the composition on plant parts, stickers that promote adhesion to the plant part, penetrants that can promote contact of the active agent with interior tissues, extenders that increase the half-life of the active agent by inhibiting environmental degradation, and humectants that increase the density or drying time of sprayed compositions. Wetters/spreaders used in the compositions can include, but are not limited to, non-ionic surfactants, anionic surfactants, cationic surfactants, amphoteric surfactants, organo-silicate surfactants, and/or acidified surfactants. Stickers used in the compositions can include, but are not limited to, latex-based substances, terpene/pinolene, and pyrrolidone-based substances. Penetrants can include mineral oil, vegetable oil, esterified vegetable oil, organo-silicate surfactants, and acidified surfactants. Extenders used in the compositions can include, but are not limited to, ammonium sulphate, or menthene-based substances. Humectants used in the compositions can include, but are not limited to, glycerol, propylene glycol, and diethyl glycol. Adjuvants that improve ease of product application include, but are not limited to, acidifying/buffering agents, anti-foaming/de-foaming agents, compatibility agents, drift-reducing agents, dyes, and water conditioners. Anti-foaming/de-foaming agents used in the compositions can include, but are not limited to, dimethopolysiloxane. Compatibility agents used in the compositions can include, but are not limited to, ammonium sulphate. Drift-reducing agents used in the compositions can include, but are not limited to, polyacrylamides, and polysaccharides. Water conditioners used in the compositions can include, but are not limited to, ammonium sulphate.

Methods of treating plants and/or plant parts with the fermentation broths, fermentation broth products, and compositions comprising yield enhancing Methylobacterium are also provided herein. Treated plants, and treated plant parts obtained therefrom, include, but are not limited to, corn. Corn plant parts that are treated include, but are not limited to, leaves, stalks, primary roots, nodal roots, seeds, fruit, tassels, silks, husks, sheaths, shanks, coleoptiles, and the like. Seeds or other propagules of any of the aforementioned corn plants can be treated with the fermentation broths, fermentation broth products, fermentation products, and/or compositions provided herein.

In certain embodiments, plants and/or plant parts are treated by applying the fermentation broths, fermentation broth products, fermentation products, and compositions that comprise yield enhancing Methylobacterium as a spray. Such spray applications include, but are not limited to, treatments of a single plant part or any combination of plant parts. Spraying can be achieved with any device that will distribute the fermentation broths, fermentation broth products, fermentation products, and compositions to the plant and/or plant part(s). Useful spray devices include a boom sprayer, a hand or backpack sprayer, crop dusters (i.e. aerial spraying), and the like. Spraying devices and or methods providing for application of the fermentation broths, fermentation broth products, fermentation products, and compositions to either one or both of the adaxial surface and/or abaxial surface can also be used. Plants and/or plant parts that are at least partially coated with any of a biphasic fermentation broth, a fermentation broth product, fermentation product, or compositions that comprise a solid substance with yield enhancing Methylobacterium adhered thereto are also provided herein. In certain embodiments, the plant part is a seed. Partial coating of a plant or a plant part includes, but is not limited to coating at least about 5%, 10%, 20%, 30%, 40%, 50%, 60%, 70%, 80%, 90%, 95%, 98%, 99%, or about 99.5% of the surface area of the plant or plant part. In some embodiments, the plant part is a seed and partial coating includes, but is not limited to coating at least about 5%, 10%, 20%, 30%, 40%, 50%, 60%, 70%, 80%, 90%, 95%, 98%, 99%, or about 99.5% of the surface area of the seed. Also provided herein are processed plant products that comprise a solid substance with yield enhancing Methylobacterium adhered thereto.

In certain embodiments, seeds are treated by exposing the seeds to the fermentation broths, fermentation broth products, fermentation products, and compositions that comprise yield enhancing Methylobacterium Seeds can be treated with the fermentation broths, fermentation broth products, and compositions provided herein by methods including, but not limited to, imbibition, coating, spraying, and the like. Seed treatments can be effected with both continuous and/or a batch seed treaters. In certain embodiments, the coated seeds can be prepared by slurrying seeds with a coating composition containing a fermentation broth or fermentation broth product comprising yield enhancing Methylobacterium and air drying the resulting product. Air drying can be accomplished at any temperature that is not deleterious to the seed or the Methylobacterium, but the temperature will typically not be greater than 30 degrees Centigrade. The proportion of coating that comprises a solid substance and yield enhancing Methylobacterium includes, but is not limited to, a range of 0.1 to 25% by weight of the seed, 0.5 to 5% by weight of the seed, and 0.5 to 2.5% by weight of seed. In certain embodiments, a solid substance used in the seed coating or treatment will have yield enhancing Methylobacterium adhered thereon. In certain embodiments, a solid substance used in the seed coating or treatment will be associated with yield enhancing Methylobacterium and will be present in a fermentation broth, fermentation broth product, or composition obtained by the methods provided herein. Various seed treatment compositions and methods for seed treatment disclosed in U.S. Pat. Nos. 5,106,648, 5,512,069, and 8,181,388 are incorporated herein by reference in their entireties and can be adapted for use with an active agent comprising the fermentation broths, fermentation broth products, or compositions provided herein. In certain embodiments, the composition used to treat the seed can contain agriculturally acceptable excipients that include, but are not limited to, woodflours, clays, activated carbon, diatomaceous earth, fine-grain inorganic solids, calcium carbonate and the like. Clays and inorganic solids that can be used with the fermentation broths, fermentation broth products, or compositions provided herein include, but are not limited to, calcium bentonite, kaolin, china clay, talc, perlite, mica, vermiculite, silicas, quartz powder, montmorillonite and mixtures thereof. Agriculturally acceptable adjuvants that promote sticking to the seed that can be used include, but are not limited to, polyvinyl acetates, polyvinyl acetate copolymers, hydrolyzed polyvinyl acetates, polyvinylpyrrolidone-vinyl acetate copolymer, polyvinyl alcohols, polyvinyl alcohol copolymers, polyvinyl methyl ether, polyvinyl methyl ether-maleic anhydride copolymer, waxes, latex polymers, celluloses including ethylcelluloses and methylcelluloses, hydroxy methylcelluloses, hydroxypropylcellulose, hydroxymethylpropylcelluloses, polyvinyl pyrrolidones, alginates, dextrins, malto-dextrins, polysaccharides, fats, oils, proteins, karaya gum, jaguar gum, tragacanth gum, polysaccharide gums, mucilage, gum arabics, shellacs, vinylidene chloride polymers and copolymers, soybean-based protein polymers and copolymers, lignosulfonates, acrylic copolymers, starches, polyvinylacrylates, zeins, gelatin, carboxymethylcellulose, chitosan, polyethylene oxide, acrylamide polymers and copolymers, polyhydroxyethyl acrylate, methylacrylamide monomers, alginate, ethylcellulose, polychloroprene and syrups or mixtures thereof. Other useful agriculturally acceptable adjuvants that can promote coating include, but are not limited to, polymers and copolymers of vinyl acetate, polyvinylpyrrolidone-vinyl acetate copolymer and water-soluble waxes. Various surfactants, dispersants, anticaking-agents, foam-control agents, and dyes disclosed herein and in US Patent No. 8, 181,388 can be adapted for use with an active agent comprising the fermentation broths, fermentation broth products, or compositions provided herein.

Provided herein are compositions that comprise yield enhancing Methylobacterium that provide for increase yield of corn plants relative to untreated plants, plant parts, and plants obtained therefrom that have not been exposed to the compositions. In certain embodiments, plant parts, including, but not limited to, a seed, a leaf, a flower, a stem, a root, or a coleoptile can be treated with the compositions provided herein to increase corn plant yield. Treatments or applications can include, but are not limited to, spraying, coating, partially coating, immersing, and/or imbibing the plant or plant parts with the compositions provided herein. Partial coating of a corn plant or a corn plant part includes, but is not limited to coating at least about 5%, 10%, 20%, 30%, 40%, 50%, 60%, 70%, 80%, 90%, 95%, 98%, 99%, or about 99.5% of the surface area of the corn plant or corn plant part. In some embodiments, the corn plant part that is partially coated is a corn plant seed. In certain embodiments, a seed, a leaf, a flower, a stem, a root, a cob, or a coleoptile can be immersed and/or imbibed with a liquid, semi-liquid, emulsion, or slurry of a composition provided herein. Such seed immersion or imbibition can be sufficient to provide for increased yield in a treated corn plant or corn plant grown from a treated seed in comparison to an untreated corn plant or corn plant grown from an untreated seed. In certain embodiments, plant seeds can be immersed and/or imbibed for at least 1, 2, 3, 4, 5, or 6 hours. Such immersion and/or imbibition can, in certain embodiments, be conducted at temperatures that are not deleterious to the plant seed or the Methylobacterium. In certain embodiments, the seeds can be treated at about 15 to about 30 degrees Centigrade or at about 20 to about 25 degrees Centigrade. In certain embodiments, seed imbibition and/or immersion can be performed with gentle agitation. In certain embodiments, the seed and/or coleoptile is exposed to the composition by providing the composition in furrow. Providing the composition in furrow represents one of several methods provided herein for applying a composition to a corn seed or to a corn plant at or prior to the VE stage of corn plant development.

Compositions provided herein comprising yield enhancing Methylobacterium and related methods are therefore expected to be useful in improving yield in a wide variety of corn plants, including, but not limited to, various Zea mays hybrids, inbreds, haploids, subspecies, and varieties. In certain embodiments, yield can be improved in dent corn (Zea mays var. indentata), flint corn (Zea mays var. indurata), flour corn (Zea mays var. amylacea), popcorn (Zea maysvar. everta), pod corn (Zea mays var. tunicata Larrañaga ex A. St. Hil.) striped maize (Zea mays var. japonica), sweet corn (Zea mays var. saccharata and Zea mays var. rugosa), and/or waxy corn (Zea mays var. ceratina).

In certain embodiments, an amount of a composition provided herein that is sufficient to provide for increased corn yield can be a composition with yield enhancing Methylobacterium at a titer of at least about 1×10⁶ colony-forming units per milliliter, at least about 5×10⁶ colony-forming units per milliliter, at least about 1×10⁷ colony-forming units per milliliter, at least about 5×10⁸ colony-forming units per milliliter, at least about 1×10⁹ colony-forming units per milliliter, at least about 1×10¹⁰ colony-forming units per milliliter, or at least about 3×10¹⁰ colony-forming units per milliliter. In certain embodiments, an amount of a composition provided herein that is sufficient to provide for increased corn yield to a plant or plant part can be a composition with yield enhancing Methylobacterium at a titer of about least about 1×10⁶ colony-forming units per milliliter, at least about 5×10⁶ colony-forming units per milliliter, at least about 1×10⁷ colony-forming units per milliliter, or at least about 5×10⁸ colony-forming units per milliliter to at least about 6×10¹⁰ colony-forming units per milliliter of a liquid or an emulsion. In certain embodiments, an amount of a composition provided herein that is sufficient to provide for increased corn yield can be a fermentation broth product with a yield enhancing Methylobacterium titer of a solid phase of that product is at least about 1×10⁶ colony-forming units per milliliter, at least about 5×10⁶ colony-forming units per milliliter, at least about 1×10⁷ colony-forming units per milliliter, or at least about 5×10⁸ colony-forming units per gram to at least about 6×10¹⁰ colony-forming units of Methylobacterium per gram of the solid phase. In certain embodiments, an amount of a composition provided herein that is sufficient to provide for increased corn yield can be a composition with a Methylobacterium titer of at least about 1×10⁶ colony-forming units per gram, at least about 5×10⁶ colony-forming units per gram, at least about 1×10⁷ colony-forming units per gram, or at least about 5×10⁸ colony-forming units per gram to at least about 6×10¹⁰ colony-forming units of Methylobacterium per gram of particles in the composition containing the particles that comprise a solid substance wherein a mono-culture or co-culture of yield enhancing Methylobacterium is adhered thereto. In certain embodiments, an amount of a composition provided herein that is sufficient to provide for increased corn yield to a plant or plant part can be a composition with a Methylobacterium titer of at least about 1×10⁶ colony-forming units per mL, at least about 5×10⁶ colony-forming units per mL, at least about 1x107 colony-forming units per mL, or at least about 5×10⁸ colony-forming units per mL to at least about 6×10¹⁰ colony-forming units of Methylobacterium per mL in a composition comprising an emulsion wherein a mono-culture or co-culture of a yield enhancing Methylobacterium adhered to a solid substance is provided therein or grown therein. In certain embodiments, an amount of a composition provided herein that is sufficient to provide for increased corn yield to a plant or plant part can be a composition with a Methylobacterium titer of at least about 1×10⁶ colony-forming units per mL, at least about 5×10⁶ colony-forming units per mL, at least about 1×10⁷ colony-forming units per mL, or at least about 5×10⁸ colony-forming units per mL to at least about 6×10¹⁰ colony-forming units of Methylobacterium per mL of in a composition comprising an emulsion wherein a mono-culture or co-culture of a yield enhancing Methylobacterium is provided therein or grown therein. In certain embodiments of any of the aforementioned compositions, the Methylobacterium is selected from the group consisting of (i) Methylobacterium isolate NLS0807 (NRRL B-67743), NLS0662 (NRRL B-67742), NLS0648 (NRRL B-67741), NLS0109 (NRRL B-67340), or variants thereof, or (ii) a combination of Methylobacterium isolate NLS0109 (NRRL B-67340) or a variant thereof, and Methylobacterium isolate NLS0017 (B-50931) or a variant thereof.

In certain embodiments, an amount of a composition provided herein that is sufficient to provide for increased corn yield can be a composition with a Methylobacterium at a titer of at least about 1×10⁴ colony-forming units per milliliter, at least about 1×10⁵ colony-forming units per milliliter, at least about 1×10⁶ colony-forming units per milliliter, at least about 5×10⁶ colony-forming units per milliliter, at least about 1×10⁷ colony-forming units per milliliter, at least about 5×10⁸ colony-forming units per milliliter, at least about 1×10⁹ colony-forming units per milliliter, at least about 1×10¹⁰ colony-forming units per milliliter, or at least about 3×10¹⁰ colony-forming units per milliliter. In certain embodiments, an amount of a composition provided herein that is sufficient to provide for increased corn yield can be a composition with Methylobacterium at a titer of at least about 1×10⁴ colony-forming units per milliliter, at least about 1×10⁵ colony-forming units per milliliter, about least about 1×10⁶ colony-forming units per milliliter, at least about 5×10⁶ colony-forming units per milliliter, at least about 1×10⁷ colony-forming units per milliliter, or at least about 5×10⁸ colony-forming units per milliliter to at least about 6×10¹⁰ colony-forming units per milliliter of a liquid or an emulsion. In certain embodiments, an amount of a composition provided herein that is sufficient to provide for increased corn yield can be a fermentation broth product with a Methylobacterium titer of a solid phase of that product is at least about 1×10⁴ colony-forming units per gram, at least about 1×10⁵ colony-forming units per gram, at least about 1×10⁶ colony-forming units per gram, at least about 5×10⁶ colony-forming units per gram, at least about 1×10⁷ colony-forming units per gram, or at least about 5×10⁸ colony-forming units per gram to at least about 6×10¹⁰ colony-forming units of Methylobacterium per gram, at least about 1×10¹¹ colony-forming units of Methylobacterium per gram, at least about 1×10¹² colony-forming units of Methylobacterium per gram, at least about 1×10¹³ colony-forming units of Methylobacterium per gram, or at least about 5×10¹³ colony-forming units of Methylobacterium per gram of the solid phase. In certain embodiments, an amount of a composition provided herein that is sufficient to provide for increased corn yield can be a composition with a Methylobacterium titer of at least about 1×10⁶ colony-forming units per gram, at least about 5×10⁶ colony-forming units per gram, at least about 1×10⁷ colony-forming units per gram, or at least about 5×10⁸ colony-forming units per gram to at least about 6×10¹⁰ colony-forming units of Methylobacterium per gram, at least about 1×10¹¹ colony-forming units of Methylobacterium per gram, at least about 1×10¹² colony-forming units of Methylobacterium per gram, at least about 1×10¹³ colony-forming units of Methylobacterium per gram, or at least about 5×10¹³ colony-forming units of Methylobacterium per gram of particles in the composition containing the particles that comprise a solid substance wherein a mono-culture or co-culture of Methylobacterium is adhered thereto. In certain embodiments, an amount of a composition provided herein that is sufficient to provide for increased corn yield can be a composition with a Methylobacterium titer of at least about 1×10⁶ colony-forming units per mL, at least about 5×10⁶ colony-forming units per mL, at least about 1×10⁷ colony-forming units per mL, or at least about 5×10⁸ colony-forming units per mL to at least about 6×10¹⁰ colony-forming units of Methylobacterium per mL in a composition comprising an emulsion wherein a mono-culture or co-culture of a Methylobacterium adhered to a solid substance is provided therein or grown therein. In certain embodiments, an amount of a composition provided herein that is sufficient to provide for increased corn yield can be a composition with a Methylobacterium titer of at least about 1×10⁶ colony-forming units per mL, at least about 5×10⁶ colony-forming units per mL, at least about 1×10⁷ colony-forming units per mL, or at least about 5×10⁸ colony-forming units per mL to at least about 6×10¹⁰ colony-forming units of Methylobacterium per mL of in a composition comprising an emulsion wherein a mono-culture or co-culture of a Methylobacterium is provided therein or grown therein. In certain embodiments of any of the aforementioned compositions, the Methylobacterium is Methylobacterium isolate NLS0807 (NRRL B-67743), NLS0662 (NRRL B-67742), NLS0648 (NRRL B-67741), NLS0109 (NRRL B-67340), or variants thereof, or (ii) a combination of Methylobacterium isolate NLS0109 (NRRL B-67340) or a variant thereof, and Methylobacterium isolate NLS0017 (B-50931) or a variant thereof. Also provided are corn plants and corn plant parts (e.g. seeds) that are coated or partially coated with any of the aforementioned compositions. Also provided are methods for improving corn yield by using any of the aforementioned compositions.

### EXAMPLES

The following examples are included to demonstrate illustrative, non-limiting embodiments of the disclosure. It will be appreciated by those of skill in the art that the techniques disclosed in the following examples represent techniques discovered by the Applicants to function well in the practice of the invention. However, those of skill in the art should, in light of the instant disclosure, appreciate that many changes can be made in the specific embodiments that are disclosed, while still obtaining like or similar results, without departing from the scope of the disclosure.

### EXAMPLE 1. INCREASES IN CORN YIELD BY APPLICATION OF METHYLOBACTERIUM COMPOSITIONS IN 2018 FIELD TRIALS

In 2018, corn field trials were established at twelve locations for the purpose of evaluating 4 PPFM (pink-pigmented-facultative-methylotrophs of the species Methylobacterium ) isolates and a combination of two different Methylobacterium isolates applied in-furrow at planting to corn plants. The trial at Frankfort, IN was not included in the corn foliar trial analysis due a portion of the trial experiencing damage.

### Experimental Design

The trial was established using a Randomized Complete Block Design (RCBD) with 4 reps per location.

### Methods

In preparation for the field trials, the PPFM cultures were grown in AMS+glycerol+peptone+diatomaceous earth, at 30° C. for 6 days. The AMS medium contains, per liter, 700 milligrams of dibasic potassium phosphate anhydrous, 540 milligrams of monobasic potassium phosphate anhydrous, one gram of magnesium sulfate heptahydrate, 500 milligrams of ammonium chloride anhydrous, and 200 milligrams of calcium chloride dihydrate.

AMS base medium was prepared from three stock solutions, listed below:

Stock solution I: for one liter at 50X concentration

| | |
|---|---|
| dibasic potassium phosphate, anhydrous | 35 grams |
| monobasic potassium phosphate, anhydrous | 27 grams |

Stock solution II: for one liter at 50X concentration

| | |
|---|---|
| magnesium sulfate heptahydrate | 50 grams |
| ammonium chloride, anhydrous | 25 grams |

Stock solution III: for one liter at 50X concentration

| | |
|---|---|
| calcium chloride dihydrate | 10 grams |

Stock solutions I, II, and III were autoclaved separately.

To prepare one liter of liquid AMS medium with glycerol, peptone, and diatomaceous earth, the following were added to 920 ml of distilled water:
20 ml of stock solution I
20 ml of stock solution II
20 ml of stock solution III
20 ml of a 50% glycerol stock solution
10 grams of peptone
2 grams of diatomaceous earth

The resulting solution with suspended diatomaceous earth was sterilized by autoclaving. The cultures were harvested by centrifugation at 5000 rpm for 15 minutes and then re-suspended in AMS+glycerol+peptone with 20% glycerol as a cryoprotectant at 10× concentration. The cultures were aliquoted and frozen at −80 until thawed for use. The liquid PPFM preparations were made in-furrow.

### Results

The analysis was conducted with JMP14 (SAS Institute), and the assumptions of normality and equal variance are tested prior to executing the Analysis of variance (ANOVA). ANOVA was conducted with the Analyze-Fit Model routine. Comparisons of isolates with the check within the same growth stage at application were performed with two-tailed t-tests applied to the pairwise differences between least-squares means estimated from the ANOVA model, under the null hypothesis that the difference in means was zero.

Across-locations analyses for the eleven locations were conducted according to the following model:

$${Y}_{h i j k} = M + {I}_{i} + {S}_{j} + I {S}_{i j} + {L}_{h} + {R ( L )}_{k ( h )} + {LI}_{h i} + I {R ( L )}_{ik ( h )} + {LS}_{h j} + L I {S}_{h i j} + {e}_{hijk} , \qquad [ 2 ]$$

where Yₕᵢⱼₖ is the yield at location h of isolate i at stage j in replicate k, M represents the overall mean, Iᵢ is the fixed effect of isolate i, Sⱼ is the fixed effect of stage j, ISᵢⱼ is the fixed effect of the interaction of isolate i and stage j, Lₕ is the random effect of location h, R(L)ₖ₍ₕ₎ is the random effect of replicate k nested within location h, LIₕᵢ is the random effect of the interaction of location h and isolate i, IR(L)ᵢₖ₍ₕ₎ is the random effect of the interaction of isolate i and replicate k nested within location h, LSₕⱼ is the random effect of the interaction of location h and stage j, LISₕᵢⱼ is the random effect of the three-way interaction of location h with isolate i and stage j, and eₕᵢⱼₖ is the random error.

The 4 PPFM isolates and the combination of isolates NLS0109 and NLS0017 tested in 2018 showed a higher yield response as compared to the untreated control.

TABLE 2
Mean yield in 2018 (Bold italics indicates a significant difference
at p < 0.05 using Fisher's LSD test.)

| | | Yield | Yield | p-value v. | Win rate |
|---|---|---|---|---|---|
| | Mean | difference | difference | UTC | (# locs |
| | yield | from UTC | from UTC | (Fisher's | above |
| Treatment | (Bu/A) | (Bu/A) | (%) | LSD test) | UTC) |
| NLS0807 | 220.5 | +6.3 | +2.9% | 0.044 | 8/11 (73%) |
| NLS0662 | 218.1 | +3.9 | +1.8% | 0.21 | 7/11 (64%) |
| NLS0648 | 217.5 | +3.3 | +1.5% | 0.29 | 7/11 (64%) |
| NLS0109 | 216.9 | +2.7 | +1.3% | 0.39 | 7/11 (64%) |
| NLS0109 + | 218.6 | +4.4 | +2.1% | 0.16 | 8/11 (73%) |
| NLS0017 | | | | | |
| UTC | 214.2 | — | — | — | — |

EXAMPLE 2. INCREASES IN CORN YIELD BY APPLICATION OF METHYLOBACTERIUM COMPOSITIONS IN 2017 FIELD TRIALS

This trial assessed yield enhancement by Methylobacterium treatments compared to 2 UTC treatments. The Methylobacterium was applied as seed treatments with a target seed titer of 10⁶ CFUs/seed for single isolate treatments. It included 6 locations. The trial was established using a Randomized Complete Block Design (RCBD) with 4 reps per location.

### The Experimental Design and Methods were the Same as Example 1.

All 4 Methylobacterium treatments produced higher yield than the UTC. The two UTC treatments (UTC-1 and UTC-2) showed very similar yields and were the two lowest-yielding treatments in the trial.

TABLE 3
Mean Yield in 2017. (P-values are taken from Dunnett's test.)

| | | Absolute | | | |
|---|---|---|---|---|---|
| | | difference | % | Contrast p- | Locations |
| | | from UTC | difference | value v. | above |
| Inoculation | Yield | mean | from UTC | UTC mean | UTC |
| treatment | (Bu/A) | (Bu/A) | mean | (Dunnett's) | (/6) |
| NLS0648 | 208.7 | +5.6 | +2.7% | 0.56 | 4 |
| NLS0662 | 212.8 | +9.7 | +4.8% | 0.11 | 6 |
| NLS0807 | 210.5 | +7.4 | +3.7% | 0.35 | 5 |
| NLS0109 + | 212.3 | +9.2 | +4.5% | 0.15 | 5 |
| NLS0017 | | | | | |
| UTC-1 | 203.7 | — | — | — | — |
| UTC-2 | 202.5 | — | — | — | — |
| UTC mean | 203.1 | — | — | — | — |

EXAMPLE 3. EFFECTS OF METHYLOBACTERIUM STRAIN NLS0807 (NRRL B-67743) TREATMENT OF CORN ON NUTRIENT CONTENT

Corn seeds were treated with Methylobacterium strain NLS0807 at a rate of 10⁶ CFU per seed and grown in sterilized soil (30 seeds per flat) in a greenhouse in parallel with untreated corn seeds. At 22 days after planting (V2 growth stage), 15 or more plants per flat were chosen randomly and shoots were collected by cutting one inch above the soil line. The shoots were incubated in sample bags at 45° C. for 4 days to dry and analyzed for macronutrient and micronutrient content. A single-tailed unequal variances (Welch's) t-test was used to analyze the data to determine whether treatment with NLS0807 resulted in a significant increase in nutrient content. Methylobacterium NLS0807 significantly enhanced foliar content of four nutrients: phosphorus (P), potassium (K), iron (Fe) and manganese (Mn). Of the 12 nutrients tested, 9 were elevated over the UTC by treatment with NLS0807. Results are shown in Table 4.

TABLE 4
Mean nutrient contents on a dry weight basis, percent
difference of NLS0807 from UTC, and contrast p-
values. (Bold italics indicate p < 0.05.)

| Nutrient | NLS0807 | UTC | % difference | Contrast |
|---|---|---|---|---|
| (units) | value | value | from UTC | p-value |
| Nitrogen (%) | 1.466 | 1.465 | +0.1% | 0.49 |
| NO3—N (%) | 0.0024 | 0.0021 | +14.3% | 0.17 |
| Phosphorus (%) | 0.227 | 0.201 | +12.9% | 0.0062 |
| Potassium (%) | 5.20 | 4.90 | +6.1% | 0.027 |
| Calcium (%) | 0.839 | 0.815 | +2.9% | 0.28 |
| Magnesium (%) | 0.254 | 0.251 | +1.2% | 0.38 |
| Sulfur (%) | 0.216 | 0.226 | −4.4% | 0.80 |
| Zinc (ppm) | 22.8 | 21.6 | +5.6% | 0.15 |
| Manganese (ppm) | 136.3 | 122.3 | +11.4% | 0.0092 |
| Iron (ppm) | 89.3 | 75.6 | +18.1% | 0.028 |
| Copper (ppm) | 6.13 | 6.33 | −3.2% | 0.73 |
| Boron (ppm) | 5.47 | 5.53 | −1.1% | 0.57 |

EXAMPLE 4 DETECTION OR IDENTIFICATION OF METHYLOBACTERIUM STRAINS, VARIANTS AND DERIVATIVES

Assays are disclosed for detection or identification of specific Methylobacterium strains and closely related derivatives. Genomic DNA fragments unique to a Methylobacterium strain are identified and qPCR Locked Nucleic Acid (LNA) based assays are developed.

Genomic DNA sequences of Methylobacterium strains are compared by BLAST analysis of approximately 300bp fragments using a sliding window of from 1-25 nucleotides to whole genome sequences of over 1000 public and proprietary Methylobacterium isolates. Genomic DNA fragments are identified that have weak BLAST alignments, indicative of approximately 60-95% identity over the entire fragment, to corresponding fragments of a Methylobacterium of interest. Fragments from the NLS0109 genome corresponding to the identified weak alignment regions were selected for assay development and are provided as SEQ ID NOS: 1-3.

TABLE 5
Target Fragment Sequences of NLS0109

| | SEQ | |
|---|---|---|
| | ID | |
| Fragment | NO | Sequence |
| ref1_135566 | 1 | ACGGTCACCCCACGGACTGGGCGAGTACCTCACCGGTGTTCTA |
| | | TCATAACGCCGAGTTAGTTTTCGACCGTCCCTTATGCGATGTA |
| | | CCACCGGTGTCGGCAGCCGATTTCGTCCCACCGGGAGCTGGCG |
| | | TTCCGGTTCAGACCACCATCATCGGTCACGATGTCTGGATTGG |
| | | ACACGGGGCCTTCATCTCCCCCGGCGTGACTATAGGAAACGGC |
| | | GCGATCGTCGGGGCCCAGGCGGTCGTCACAAGAGATGTCCCA |
| | | CCCTATGCGGTAGTTGCTGGCGTCCCCGCGACCGTACGACGAT |
| ref1_135772 | 2 | CCAATAAAAGCGTTGGCCGCCTGGGCAACCCGATCCGAGCCT |
| | | AAGACTCAAAGCGCAAGCGAACACTTGGTAGAGACAGCCCGC |
| | | CGACTACGGCGTTCCAGCACTCTCCGGCTTTGATCGGATAGGC |
| | | ATTGGTCAAGGTGCCGGTGGTGATGACCTCGCCCGCCGCAAGC |
| | | GGCGAATTACTCGGATCAGCGGCCAGCACCTCGACCAAGTGT |
| | | CGGAGCGCGACCAAAGGGCCACGTTCGAGGACGTTTGAGGCG |
| | | CGACCAGTCTCGATAGTCTCATCGTCGCGGCGAAGCTGCACCT |
| | | CGA |
| ref1_169470 | 3 | CGATGGCACCGACCTGCCATGCCTCTGCCGTCCGCGCCAGAAT |
| | | GGTAAAGAGGACGAAGGGGGTAAGGATCGTCGCTGCAGTGTT |
| | | GAGCAGCGACCAGAGAAGGGGGCCGAACATCGGCATCAAACC |
| | | TCGATTGCCACTCGGACGCGAAGCGCGTCTTGAAGGAGGGAT |
| | | GGAAGCGAAACGGCCGCAGAGTAACCGCCGACGAAAGATTGC |
| | | ACCCCTCATCGAGCAGGATCGGAGGTGAAGGCAAGCGTGGGT |
| | | TATTGGTAAGTGCAAAAAATATAATGGTAGCGTCAGATCTAGC |
| | | GTTC |

Regions in SEQ ID NOS: 1-3 where corresponding regions in other Methylobacterium strains were identified as having one or more nucleotide mismatches from the NLS0109 sequence were selected, and qPCR primers designed using Primer3 software (Untergasser et al. (2012), Koressaar et al. (2007)) to flank the mismatch regions, have a melting temperature (Tm) in the range of 55-60 degrees, and to generate a PCR DNA fragment of approximately 100 bp. The probe sequence was designed with a 5′ FAM reporter dye, a 3′ Iowa Black FQ quencher, and contains one to six LNA bases (Integrated DNA Technologies, Coralville, Iowa). At least 1 of the LNA bases is in the position of a mismatch, while the other LNA bases are used to raise the Tm. The Tm of the probe sequence is targeted to be 10 degrees above the Tm of the primers.

Primer and probe sequences for detection of specific detection of NLS0109 are provided as SEQ ID NOS: 4-12 in Table 6. Each of the probes contains a 5′ FAM reporter dye and a 3′ Iowa Black FQ quencher.

TABLE 6
Primer and Probe Sequences for Specific Detection of NLS0109

| | SEQ | |
|---|---|---|
| Primer/Probe | ID NO | Sequence* |
| NLS0109_ref1_135566_forward | 4 | CCTCACCGGTGTTCTATCATAAC |
| NLS0109_ref1_135566_reverse | 5 | CCGATGATGGTGGTCTGAAC |
| NLS0109_ref1_135566_probe | 6 | CGTCCCTTATGCGATGTACCA |
| NLS0109_ref1_135772_forward | 7 | GATCCGAGCCTAAGACTCAAAG |
| NLS0109_ref1_135772_reverse | 8 | GACCAATGCCTATCCGATCAA |
| NLS0109_ref1_135772_probe | 9 | AACACTTGGTAGAGACAGCC |
| NLS0109_ref1_169470_forward | 10 | AAGGAGGGATGGAAGCGAAAC |
| NLS0109_ref1_169470_reverse | 11 | ATAACCCACGCTTGCCTTC |
| NLS0109_ref1_169470_probe | 12 | CGCAGAGTAACCGCCGACGAA |

*Bold and underlined letters represent the position of an LNA base

### Use of Primer/Probe Sets on Isolated DNA to Detect NLS0109 and Distinguish from Related Methylobacterium Isolates

Each 10 μl qPCR reaction contains 5 μl of Quantabio PerfeCTa qPCR ToughMix 2×Mastermix, Low ROX from VWR, 0.5 μl of 10 μM forward primer, 0.5 μl of 10 μM reverse primer, 1 μl of 2.5 μM probe, 1 μl nuclease free water and 2 μl of DNA template. Approximately 1 ng of DNA template is used per reaction. The reaction is conducted in a ThermoFisher QuantStudio™ 6 Flex Real-Time PCR System with the following program: 95° C. for 3 min, then 40 cycles of 95° C. for 15 sec and 60° C. for 1 min. The analysis software on the PCR instrument calculates a threshold and Ct value for each sample. Each sample was run in triplicate on the same qPCR plate. A positive result is indicated where the delta Ct between positive and negative controls is at least 5.

Use of the three primer/probe sets to distinguish NLS0109 from closely related isolates by analysis of isolated DNA is shown in Table 7 below. The similarity score shown for the related isolates takes into account both the average nucleotide identity and the alignment fraction between the isolates and NLS0109. One of the tested strains, NLS0730, was used as an additional positive control. NLS0730 is a clonal isolate of NLS109 which was obtained from a culture of NLS0109, which was confirmed by full genome sequencing as identical to NLS0109, and which scored positive in all three reactions. The similarity score of greater than 1.000 for this strain is likely the result of a slightly different assembly of the genome for this isolate compared to NLS0109. The delta Ct of approximately 15 or more between the NLS0109 and NLS0730 isolates and the water only control is consistent with the sequence confirmation of the identity of these isolates. Analysis of other isolates that are less closely related to NLS0109 results in delta Ct values similar to those for the water only control.

TABLE 7

| | Similarity | | | |
|---|---|---|---|---|
| | score to | Average Ct Value | | |
| NLS# | NLS0109 | Ref1_135566 | Ref1_135772 | Ref1_169470 |
| NLS0730 | 1.005 | 21.08 | 21.31 | 20.35 |
| NLS0109 | 1 | 21.97 | 22.62 | 22.08 |
| NLS0731 | 0.181 | No Ct | 37.85 | >37.91 |
| NLS0644 | 0.87 | >36.8 | >38.31 | No Ct |
| NLS0700 | 0.88 | >38.36 | >38.36 | >38.44 |
| NLS0710 | 0.894 | No Ct | >37.47 | >38.13 |
| NLS0834 | 0.852 | 37.81 | No Ct | 37.97 |
| NLS0939 | 0.862 | 37.94 | 38.37 | >38.35 |
| NLS0947 | 0.807 | 38.44 | No Ct | No Ct |
| NLS1015 | 0.894 | 38.77 | No Ct | >37.91 |
| NLS1217 | 0.872 | 37.64 | 37.20 | 37.96 |
| H2O only | | >38.14 | >35.92 | >37.12 |

### Use of Primer/Probes for Detection of NLS109 on Treated Plant Materials

For detection of NLS0109 foliar spray treatment on corn: Untreated corn seeds were planted in field soil in the growth chamber and watered with non-fertilized R.O. water. After plants germinated and grew for approximately 3 weeks, they were transferred to the greenhouse. At V5 stage, plants were divided into 3 groups for treatment: foliar spray of NLS0109, mock foliar spray, and untreated. Plants receiving the foliar spray of NLS0109 were treated with 10× glycerol stock at the rate of 71.4 μl per plant using Solo sprayers. This converts to the rate of 10L/acre in the field. Mock treated plants were sprayed with 71.4 μl water/plant. Untreated plants received no foliar spray treatment. Leaves were harvested two weeks after foliar spray treatment into sterile tubes and DNA from bacteria on the harvested leaves is isolated as described above. Each experiment was grown at least 2 times. As shown in Table 8, NLS0109 is detected on leaves harvested from corn plants treated by a foliar spray application of the Methylobacterium strains using all 3 primer probe sets, as demonstrated by delta Ct values of approximately 10 between the sample and the negative controls.

TABLE 8

| | Average Ct Value | | |
|---|---|---|---|
| Treatment | Ref1_135566 | Ref1_135772 | Ref1_169470 |
| Control (no | 32.43 | 32.10 | 31.55 |
| application) | | | |
| Control (mock | 35.54 | 35.34 | 34.80 |
| application) | | | |
| NLS0109 (10 L/acre | 23.36 | 22.88 | 22.66 |
| equivalent) | | | |

The above results demonstrate the use of genome specific primers and probes to detect Methylobacterium strain NLS0109 on various plant tissues following treatment with the strains and provide methods to distinguish NLS0109 from closely related isolates. Similar methods are developed for additional Methylobacterium strains, NLS0017, NLS0807, NLS0662 and NLS0648 using target sequence fragments and primer/probe pairs as shown in the Tables below.

TABLE 9
Target Fragment Sequences of NLS0017

| | SEQ | |
|---|---|---|
| | ID | |
| Fragment | NO | Sequence |
| ref4_930 | 13 | GCAAAACGACCTAATAGTTCTACAGCGGCATGCGCCAAGT |
| | | CAGCGCGGTGAACAGTATACCTGGGAGCAACTTGTCCTCC |
| | | GAAACCCACATAAAACAAATTACTCCTGGCAGTGCCCAGT |
| | | CCATCAAAATCGAATACAATATTTCTCGAGGAGGCATCTGT |
| | | AATAGCCTGCCAAAGCAACAAAGCTATGGCGCCGTTATGA |
| | | CTTTCATTGCTTCTGGTAGACATAAAATAATATGCCGATTT |
| | | GTGATCCCAAATGTAGAATATTGCCGCATCAATTGCGCCAA |
| | | GTTTATTTCGGATCGAT |
| ref1_142021 | 14 | GGCGCCAACGGTATGATCGCATGATTTTCCTGCGGCATAGC |
| | | TTGCGGGAATGGCGTATTTGGCGCTCTCCTCAGGAATTTCT |
| | | AAGGGCATACGCAGGAACTCTACAGCACTTTTACTGGTATT |
| | | TTGTAGTGACAGCGGAGGAGGCTGGTGCTCAAGGTAATCG |
| | | TGATGAAGTGATCCGGGCCATTCGGGGCGCGTTTCTAGTCT |
| | | TTCCAATCCGCGCCCTGTACCACGTATTACGCCGGACCGGT |
| | | CTGCGCCGCGCCGCCCTCTTGACCGCCCTAAATGTCTAAGA |
| | | GCGTCTAACAAAGC |
| ref1_142636 | 15 | GACGATATCGCTCATCTTCACTGCATTGAAGCTGGTGCCGT |
| | | ACTGCATAGGGATGAAAAAGTGATGCGGATAGACGGCTGA |
| | | CGGGAAAGCGCCTGGTCGATCGAAGACTTTGCTGACGAGG |
| | | TTGTGGTAGCCCCGGATATAGGCATCGAAGGCCGGGACGT |
| | | TGATCCCATCCTTTGCCTTATCTTGACTGGCGTCGTCGCGTG |
| | | CCGTCAGAACGGGCACGTCGCAGGTCATCGAGGCCAGCAC |
| | | CTTGCGGAACACCTGCGTTCCGCCGTTGGGATTATCGACGG |
| | | CGAACGCGGTGGCCGC |

TABLE 10
Primer and Probe Sequences for Specific Detection of NLS0017

| | SEQ | |
|---|---|---|
| | ID | |
| Primer/Probe | NO | Sequence* |
| NLS0017_ref4_930_forward | 16 | GTCCTCCGAAACCCACATAAA |
| NLS0017_ref4_930_reverse | 17 | CTACCAGAAGCAATGAAAGTCAT |
| NLS0017_ref4_930_probe | 18 | TCTGTAATAGCCTGCCAAAGCA |
| NLS0017_ref1_142021_forward | 19 | GGCTGGTGCTCAAGGTAAT |
| NLS0017_ref1_142021_reverse | 20 | ACATTTAGGGCGGTCAAGAG |
| NLS0017_ref1_142021_probe | 21 | ATGAAGTTGATCCGGGCCAT |
| NLS0017_ref1_142636_forward | 22 | CCGTACTGCATAGGGATGAAA |
| NLS0017_ref1_142636_reverse | 23 | TAAGGCAAAGGATGGGATCAA |
| NLS0017_ref1_142636_probe | 24 | TTGCTGACGAGGTTGTGGTAG |

*Bold and underlined letters represent the position of an LNA base

TABLE 11
Target Fragment Sequences of NLS0807

| | SEQ | |
|---|---|---|
| | ID | |
| Fragment | NO | Sequence |
| ref1_458355 | 25 | CAACTATGTAGACCCGACGGTGCGATTTCACTTCGCAAAGCCG |
| | | CAGGGCAGCACCCTTGCGCTCAATGTTGACGCCAGCGTGATCT |
| | | ATACTATTACCGTCACGCACACGCAGGGCGGCGTACAGATTCA |
| | | TCGCGAGAGTAAGAACCACCATCAGACCATCACGCGCAGCGA |
| | | CCTGAGCAAGCAGTTCGGCGTTGGTGTGGCCGACCAGCTGAC |
| | | GCGCGATCAGGTCATGAAGGTGATCGAGTCGGCATTTCGCGA |
| | | CGCTACCCGCTAAGATCGGCGCCCACGAAACGCTACGAGACT |
| | | AGG |
| ref1_459688 | 26 | AGCCGGCATCTTGTTCAAGGCGCTCACCTCGACGCCGACGCTG |
| | | TAGGCGACTTGAGAGGGCGTCTCATATGAACGAAGCATCTTCG |
| | | CGTAGAGAACCTTCTTGTTCTCCTGCGTGATGTTCGCTTTGCAG |
| | | ACGTTGACTGCCGCCATGAACGCCGAAGCCTTGCGCGCTTCAT |
| | | CGTAATCGCCTGCGAAGGCGGGTAGTGAAAAGCTTAGTGCAA |
| | | TGGCAAACACAGCCGCCGAACGTCGCATGGTATCCGTCCCCG |
| | | ATTGACGGCAGTGCCGCCATATCTCGGCTTTAGCAGAGCTGAT |
| ref1_3158527 | 27 | AACCTGCGCCGGCCGAGGTTTCGCGAGCCGTCGCCACGGGCA |
| | | ACGCCTCGCCCGCGATGTGCAAAAAAGTCCCCGGCACTTCGCG |
| | | CCGTCGTCCGATCCACGACCGCGAATTTCTCAACGAGTACAAG |
| | | GTGCTTATGGGAGATCCGAGCGTCCGTCCCGGAGCCCGAGAC |
| | | CGCGCGGCCCGAGTAATAGGCGAAAAAGACTCCTACTCCTCG |
| | | GGCTTCTCGGGCCCCCTCAGCAACATCTACGCTTGCCGCCCAT |
| | | CACCCTGGCGGGAGATCAGCGACGAGACACAGGCCCACTTCG |
| | | CCC |

TABLE 12
Primer and Probe Sequences for Specific Detection of NLS0807

| | SEQ | |
|---|---|---|
| Primer/Probe | ID NO | Sequence* |
| NLS0807_ref1_458355_forward | 28 | TTGACGCCAGCGTGATCTATAC |
| NLS0807_ref1_458355_reverse | 29 | GTGATGGTCTGATGGTGGTTCT |
| NLS0807_ref1_458355_probe | 30 | TATTACCGTCACGCACACG |
| NLS0807_ref1_459688_forward | 31 | CTTCGCGTAGAGAACCTTCTTGTT |
| NLS0807_ref1_459688_reverse | 32 | CTTCGCAGGCGATTACGATGAA |
| NLS0807_ref1_459688_probe | 33 | CGTGATGTTCGCTTTGCAGA |
| NLS0807_ref1_3158527_forward | 34 | CCGCGAATTTCTCAACGAGTACA |
| NLS0807_ref1_3158527_reverse | 35 | GCCCGAGGAGTAGGAGTCTTT |
| NLS0807_ref1_3158527_probe | 36 | AGGTGCTTATGGGAGATCCG |

*Bold and underlined letters represent the position of an LNA base

Use of the primer/probe sets to distinguish NLS0807 from closely related isolates by analysis of isolated DNA is shown in Table 13 below. The similarity score shown for the related isolates takes into account both the average nucleotide identity and the alignment fraction between the isolates and NLS0807. Two of the tested strains, NLS0821 and NLS0044, were used as additional positive controls since a similarity score of 1.00 indicates they are nearly identical to NLS0807. Consistently low Ct values from qPCR using NLS0807 as the DNA template and no detection in the water only control is consistent with the sequence confirmation of the identity of these isolates. Analysis of other isolates that are less closely related to NLS0807 results in no detection similar to those for the water only control.

TABLE 13

| | Similarity | | | |
|---|---|---|---|---|
| | to | Average Ct Value | | |
| NLS# | NLS0807 | ref1_459688 | ref1_3158527 | ref1_458355 |
| NLS0807 | 1.00 | 22.39 | 24.09 | 23.10 |
| NLS0821 | 1.00 | 22.49 | 24.04 | 22.96 |
| NLS0044 | 1.00 | 22.49 | 23.86 | 22.90 |
| Strain A | 0.95 | UDT | UDT | UDT |
| Strain B | 0.94 | UDT | UDT | UDT |
| Strain C | 0.93 | UDT | UDT | UDT |
| Strain D | 0.93 | UDT | UDT | UDT |
| water only (neg | — | UDT | UDT | UDT |
| control) | | | | |

TABLE 14
Target Fragment Sequences of NLS0648

| | SEQ | |
|---|---|---|
| | ID | |
| Fragment | NO | Sequence |
| ref1_1185955 | 37 | AGTCATTGATCAAGCAACCCCTATTGAGTTGGATATCGAAGGA |
| | | TCAAGGTCGCGTCAATAGATGCATCTATCAGGCCAAATGTCGC |
| | | TTTTCAAGAATGGCTCTTTCGAAGCTATCTTTATAATCGCTCGC |
| | | CATTCTCTCATTACCAAAATCGACCTTAACTAGCTCGACATTG |
| | | ATGCGAGCAGCTCCGGCAAACGAGGAGAGATTGACCTTAAAG |
| | | GAATTGAACGCCTCAAGCAATTCAGACACATTACCAGGAGTG |
| | | CTATAGCAACAACCAGACCCATATCGGTCAATAACCTCTTTTA |
| ref1_3282585 | 38 | CGCAAAACGATTTATCACTGCCATCTTGTTGTTTGATAACCCTT |
| | | TTTTACCAGACGTTATGCTGGGCGAGAAAGAGGACTAGCAGA |
| | | TCGGAGCGGTATCGCGATTTTTCGGTAGTTCGCGCCTACAACA |
| | | GGATAAGATCCGATAGTGAAGCAACATGGCTGTTTTTTGATTT |
| | | GTAAGTCAGCAACTTAAGCAGCCAGCCTATCTGCCGTCGCAGA |
| | | CGCTTGAGGCATCGGGCAGCATCTTAGAAAAGGTGGCAGTAA |
| | | TTGCCACAGCGGAACGTAGCGGCACGGATAAGCACGCAGGGT |
| | | C |
| ref1_4194637 | 39 | CCCATCTGGACCCAATATCCCCTTCATCGACAATTCCCGAGTA |
| | | AGTGTGGGTTCGAGGATTTCGCGAAACAGCCTTGTTCGTTCCT |
| | | CCGGCCTTAAAATTGGCGTGCCGTCGGGAGATCGATAGGCATC |
| | | CCTTACCTGCCTTTCGACCGCCGGCACACGCGCGCCGGTCGTC |
| | | GTGTTCACGGCCACGGAATGGACGAAGGTGCGCCGCTCATTTC |
| | | GCTCGTTTGCCGTCTCCACCATCCAGGAGGCCAGCAGGACGGT |
| | | TTCGTCTCGACCGCCGGTCACACACACCGCAAGGGACTCAGG |

TABLE 15
Primer and Probe Sequences for Specific Detection of NLS0648

| | SEQ | |
|---|---|---|
| Primer/Probe | ID NO | Sequence* |
| NLS0648_ref1_1185955_forward | 40 | TCGCTCGCCATTCTCTCATTAC |
| NLS0648_ref1_1185955_reverse | 41 | AGGTCAATCTCTCCTCGTTTGC |
| NLS0648_ref1_1185955_probe | 42 | TCGACATTGATGCGAGCA |
| NLS0648_ref1_3282585_forward | 43 | TTCGCGCCTACAACAGGATAAG |
| NLS0648_ref1_3282585_reverse | 44 | CAGATAGGCTGGCTGCTTAAGTT |
| NLS0648_ref1_3282585_probe | 45 | TCCGATAGTGAAGCAACA |
| NLS0648_ref1_4194637_forward | 46 | GAGTAAGTGTGGGTTCGAGGATTT |
| NLS0648_ref1_4194637_reverse | 47 | AGGTAAGGGATGCCTATCGATCT |
| NLS0648_ref1_4194637_probe | 48 | CGGAGGAACGAACAAGGC |

*Bold and underlined letters represent the position of an LNA base

TABLE 16
Target Fragment Sequences of NLS0662

| | SEQ ID | |
|---|---|---|
| Fragment | NO | Sequence |
| NLS0662_ref1_4871392 | 49 | ACCTGCTAAAATCACGTCCTCTCAGATTGAAAAAT |
| | | CATTGAAGAAACGTGTCGAACGATTGCCGGGGATT |
| | | ATGACGTTAGATCAATTGAAAAATACAAGCTTTGA |
| | | AATTGAGTTACAGCCAAAAGATGCCCCGGATCCGG |
| | | ACCCATCAGACTTCGGTGGCTAGTTCGAGCCAAAC |
| | | TCGAACGTCGCCATGGCGCGCAAGTCGCAATACCA |
| | | TTTCACAGCGCAGCGGTTATTTCGTTGTACACTGTA |
| | | GCAATGCGTCGGCTTGCGCGCTTCCGCTGGCGATC |
| | | AAAGGTCCGCCGATTTACG |
| NLS0662_ref1_1266930 | 50 | TCCCGAACATACAATGGAGGAAGCGTGTGGTAGGC |
| | | CAATTTGTAACGAAATATGGCATCGGTCACGGCTC |
| | | TCTCAATAAATTCGATCTCAAGTCTTCTGAACGAG |
| | | CATGCCTCATCCTTATCCTGAGCGAACGCCTGCCA |
| | | GTTTGCAGTCATTCCAACATACATAGCCAAAAAGG |
| | | CGAGGTAGACCTTCATACGGGCACCTCAATCGTCC |
| | | CCATTCGTTCAAGCTCCTTCAAGATAACAGCCGCA |
| | | CCACATTGCTGAGATCGAAGATTCGGATCAAATAT |
| | | TCCATCAAATTTATACTTTC |
| NLS0662_ref1_17614 | 51 | GCATCCTTTGCGCTCGCAGGCCTAAGGTCAAGCCC |
| | | GGTTACTTCGTTTGGTAGAACGAGGTAGACGATGC |
| | | CTAGTCTTAAGGTGGCCCATGTTAACCAACAGGGC |
| | | CAGAACATGATTATAGTTCCGTTAGATGCCAACTT |
| | | CGGTTACAAAACCGATGGTGAGCAGTCCGACATCA |
| | | TGTTCGAAATACAGGACGCGGCGCGGTCCGCCGGT |
| | | CTTGCGGGTGCCGTAGTAGCGTTCTGGCAGTCAGG |
| | | TGGACAAACCCGTTTCCGGGGCCCGGCTCCGTGGC |
| | | ACCCATTCCTTCGCAGCCTC |

TABLE 17
Primer and Probe Sequences for Specific Detection of NLS0662

| Primer/Probe | SEQ ID NO | Sequence* |
|---|---|---|
| NLS0662_ref1_4871392_forward | 52 | GCGCAAGTCGCAATACCATTTC |
| NLS0662_ref1_4871392_reverse | 53 | CGTAAATCGGCGGACCTTTGA |
| NLS0662_ref1_4871392_probe | 54 | CGCAGCGGTTATTTCGTTG |
| NLS0662_ref1_1266930_forward | 55 | ACGAGCATGCCTCATCCTTATC |
| NLS0662_ref1_1266930_reverse | 56 | CGATTGAGGTGCCCGTATGAA |
| NLS0662_ref1_1266930_probe | 57 | TGCCAGTTTGCAGTCATTCC |
| NLS0662_ref1_17614_forward | 58 | CCCGGTTACTTCGTTTGGTAGAA |
| NLS0662_ref1_17614_reverse | 59 | CGAAGTTGGCATCTAACGGAACTA |
| NLS0662_ref1_17614_probe | 60 | TGGCCCATGTTAACCAACAG |

*Bold and underlined letters represent the position of an LNA base

### Use of Primer/Probes for Detection of NLS0807 on Treated Plant Materials

Detection of NLS0807 from In-Furrow Treated Corn Roots

At planting, corn seeds in soil were drenched with NLS0807 and control strains from frozen glycerol stock to simulate in-furrow treatment. To obtain a final concentration of 10⁷ CFU/seed, 100 μl of each strain at 10⁸ CFU/ml is inoculated onto each seed placed in the dibble holes in soil. A 1/10 dilution series is made for lower concentration targets. For control treatment, 100 μl Milli-Q water is applied to each corn seed placed in the dibble holes in soil. Pots containing treated seeds are placed in a growth chamber for approximately two weeks and watered with unfertilized RO water every 1-2 days to keep soil moist. After 2 weeks of growth, roots of about 9 plants per replicate sample were harvested into sterile tubes. Each treatment had at least 2 replicate samples in each experiment, and each experiment was conducted at least 3 times.

DNA from bacteria on the harvested corn roots is isolated as follows. Individual roots are submerged in 20 mL of phosphate-buffered saline (PBS) (137 mM NaCl, 10 mM Phosphate, 2.7 mM KCl, and a pH of 7.4) in 50 mL conical tubes. Tubes are vortexed for 10 minutes, and then sonicated for 10 minutes. Root tissue is removed, and the remaining supernatant from multiple roots of the same sample are combined and centrifuged at 7500xg for 10 minutes. This process is repeated until there is one tube for each sample. The moist soil pellet is vortexed until it evenly coats the tube wall. Tubes are placed into a laminar flow hood with caps removed and open ends of the tubes facing the air blowers. Once dry, samples are stored at room temperature. 250 mg dried soil is used as input for DNA extraction using Qiagen DNeasy PowerSoil HTP 96 kit (Cat #12955-4) using manufacturer protocols.

Primers and probes for NLS0807 disclosed in Table 12 above are used in qPCR reactions to detect the presence of NLS0807 specific fragments provided in Table 11. Each 10 μl qPCR reaction contains 5 μl of Quantabio PerfeCTa qPCR ToughMix 2×Mastermix, Low ROX from VWR, 0.5 μl of 10 μM forward primer, 0.5 μl of 10 μM reverse primer, 1 μl of 2.5 μM probe, 1 μl nuclease free water and 2 μl of DNA template. Approximately 1 ng of DNA template is used per reaction. The reaction is conducted in a ThermoFisher QuantStudio™ 6 Flex Real-Time PCR System with the following program: 95° C. for 3 min, then 40 cycles of 95° C. for 15 sec and 60° C. for 1 min. The analysis software on the PCR instrument calculates a threshold and Ct value for each sample. Each sample is run in triplicate on the same qPCR plate. A positive result is indicated where the delta Ct between positive and negative controls is at least 5.

### Use of Primer/Probes for Detection of Variants of Additional Table 1 Methylobacterium Isolates

Variants of Methylobacterium isolates listed in Table 1 are identified by the presence of DNA fragments as described above. Unique fragments for use in such methods are provided in Table 18.

TABLE 18

| | | SEQ | |
|---|---|---|---|
| Strain | Fragment | ID NO | Sequence |
| NLS0020 | ref3_25009 | 61 | GCCCTTCTGTCAGGCGATATTGTATAATGGCGTT |
| | | | GCCCCAATAGAAGCAGCCATTCGTGCGAGGGCA |
| | | | GCAGCGACGCTAGGTCGAAAGAGCATCCTAATCT |
| | | | CGATCAAGATGCGACTGAGATTTCTGATGAAAAT |
| | | | ATCTAGACACAAGCAAAGCTGGTGAAATTACAA |
| | | | CGATCATGGCGACAATTGCGGCCAATTCGGCCGG |
| | | | AACTTGAAGGAACATAAAAATGAATATTACAAA |
| | | | TATACCGCAAAGCATGTAGAGTTGCTACACCAAG |
| | | | GGTCGGGACGTCCAAAAAAACTCACTGAGGA |
| NLS0020 | ref3_25219 | 62 | GGAACATAAAAATGAATATTACAAATATACCGC |
| | | | AAAGCATGTAGAGTTGCTACACCAAGGGTCGGG |
| | | | ACGTCCAAAAAAACTCACTGAGGAAGTCGACTG |
| | | | GAAGCACGAGGCGCCCCCCCCAGGAGCGGGGCG |
| | | | ACCGGCAAGGGGGCCCGCAATTGTCGCCATGATC |
| | | | GACCAGCTTAGGTAGGATCCTCTTTCGACCTAAC |
| | | | GAATGGCTGCTTCTATTGGGGCAACGCCATTATA |
| | | | CAATATCGCCTGACCATCTGGAACGCGGCCCGGT |
| | | | CCACCGGCAGGTTGGCGACGACAGCGTCGGAG |
| NLS0020 | ref1_4361220 | 63 | CGGCGTCGACCAGCCGGGCGAACTGCTTGGGCAT |
| | | | GCTCTCCCGCGACGCCGGCCACAGCCGCGTCCCC |
| | | | GTCCCTCCGCACAGGATCATCGGGTGGATTTGAA |
| | | | AGGCAAAACGGGACATCAGGATAGGCCGCTCAG |
| | | | GCGTTGGCGCTGAGGCGCTTGATGTCGGCGTCGA |
| | | | CCATCTCGGTGATCAGCGCCTCGAGGCTGGTCTC |
| | | | GGCCTCCCAGCCGAAGGTCGCCTTGGCCTTGGCG |
| | | | GGGTTGCCCAGCAGCACCTCGACCTCTGCCGGCC |
| | | | GGAACAGCGCCGGGTCGACGATCAGGTGG |
| NLS0020 | ref1_4602420 | 64 | CTGGACATGCGCCCACCCCGGCCAAGTCCGACCG |
| | | | CACCGGCAACCGCTCCTGTAGTCGTCGTCATCGT |
| | | | TCTCACCCCTGAGGCGGAGACCGTCCGCTAACGG |
| | | | GGTGTCTCAAGCAACCGTGGGGCGGAGGAACAC |
| | | | GCACGTAGTCGCGTTTCAAGGTTCGCACGAACGC |
| | | | CTCGGCCATGCCGTTGCTCTGCGGGCTCTCCAGC |
| | | | GGCGTCGTTTTTGGCACCAAACCAAGGTCGCGGG |
| | | | CGAAGCGGCGCGTGTCGCGGGGACTGTCAGGAA |
| | | | TTTCGTGTGGGGGCGGCCATAGTGGATCCG |
| NLS0089 | ref1_194299 | 65 | GGAAATCGGCTTCAAGTACGACGTCACGCCGGCC |
| | | | ATGCAGGTCACGGGTGCACTGTTCAATCTCGAGC |
| | | | GCGACAACCAGCCGTTCCCCTCGAACGTGGAGTC |
| | | | CGGCCTCGTCCTTGGCGCAGGTCAGACACGCACC |
| | | | CAGGGCGCGGAAATCGGCCTGGCCGGCTATCTAA |
| | | | CCGATTGGTGGCAGGTCTTTGGCGGCTACGCTTA |
| | | | TACCGAGGCACGCGTACTCTCGCCACTGGAAGAC |
| | | | GATGGAGACGTGATCGCAGCAGGTAATCTCGTCG |
| | | | GCAACGTTCCGCTAAATACTTTCAGTCT |
| NLS0089 | ref1_194305 | 66 | CGGCCTGGCCGGCTATCTAACCGATTGGTGGCAG |
| | | | GTCTTTGGCGGCTACGCTTATACCGAGGCACGCG |
| | | | TACTCTCGCCACTGGAAGACGATGGAGACGTGAT |
| | | | CGCAGCAGGTAATCTCGTCGGCAACGTTCCGCTA |
| | | | AATACTTTCAGTCTGTTCAACAAGTTCGATATCA |
| | | | ACGAGAATTTCTCCGTTGCTCTGGGCTATTACTAT |
| | | | CAGGATGCCAGCTTTGCCTCCTCAGACAATGCAG |
| | | | TGCGTTTGCCAAGTTATTCGCGGTTCGATGGCGG |
| | | | GTTGTTCTATCGATTCGACGAGTTGAC |
| NLS0089 | ref1_194310 | 67 | ACGTTCCGCTAAATACTTTCAGTCTGTTCAACAA |
| | | | GTTCGATATCAACGAGAATTTCTCCGTTGCTCTG |
| | | | GGCTATTACTATCAGGATGCCAGCTTTGCCTCCTC |
| | | | AGACAATGCAGTGCGTTTGCCAAGTTATTCGCGG |
| | | | TTCGATGGCGGGTTGTTCTATCGATTCGACGAGT |
| | | | TGACACGCGTTCAGCTTAGCGTCGAGAACATTTT |
| | | | CGACAGGCGTTACATCATCAACTCCAACAACAAC |
| | | | AACAACCTCACGCCTGGCGCGCCGAGAACAGTCC |
| | | | GCGTGCAATTGATCGCTCGGTTCTAAA |
| NLS0042 | ref1_86157 | 68 | AGCCCACAAGCCTGATGCACTTAACTACATCCTC |
| | | | TAATGTCGCGCCAATTTGCTTGGCGGCAGGGGAT |
| | | | GTTGTATCGTCATAGGCTTGTCTAACCGGAACTT |
| | | | GTTTGCCAATCTCTTTGGCGATCGCAACCGCCAT |
| | | | CTCGTGTTCGTCAACCATGTGCGCGTTCCTCTAAT |
| | | | TGCACTCATGGTGCCACGTGCACCTCCGATCGTC |
| | | | TCGTGTCTAGAATGAAGGTGGGAACAACCTTACA |
| | | | CAGGCTTTCGCGACGCGCGAATTTCTGGTTTCTCC |
| | | | GCCTCGGATGTGGGTTTGAGCGCTTC |
| NLS0042 | ref1_142469 | 69 | CTTTTCATTTGTCATGATCTCGACCAAGGTATTCA |
| | | | CGGCAAGCTCGGTCTGTTGCTTAGCAAGTGCCTG |
| | | | AACTTCGCGAACGATCGGCTCTCGACCCTTCGGG |
| | | | TTCGAGACCTGTCCCTTTTGAAAACCACGTGCCC |
| | | | TACACTTTTCGGGATCAAGGTGCGGGTTGGCTTT |
| | | | GGTCAAAATTCTCTGGCGTCCCATTACACGCCCT |
| | | | CCGCATCATCGTTCCCGCGAACGATCTGACCCCC |
| | | | GACTTCCGCGAGGAAGCGTGTGGCGTGATCCTCG |
| | | | AAGCGGAATGCCACCTCGAACTGTTCC |
| NLS0042 | ref1_142321 | 70 | CAGCAGCAAGCAGATCGTTGAAAACCGCTTGAA |
| | | | CCGCATCTTGATCGGGACCGGAACCAATCAGGTC |
| | | | ATCTAGGTAAACCGAGACGTAAACTCGTTTGCGC |
| | | | TCGGCATCTTTCAGAACGTCCGTGATGCCAGACC |
| | | | GCATTAGTACCATCGTCGCCAAGGCGGGCGACTG |
| | | | AACGAAGCCGATCGGCAGAGAGTAACGGGGACC |
| | | | GCCCCTAATCGGGTTGCGAACGCAAGACCACTTA |
| | | | GCAAAGGTTCGAGCACGGCCGAACTTCGCATGGT |
| | | | GGAGAGCCGCGGCAACACGGTTCCGTGATA |
| NLS0064 | ref1_153668 | 71 | TAGACATTCCAACAAACCGGCAAGAGGCTCGTCC |
| | | | TCACTCGAGGATTTGTTGGGACTTGCATGATGTC |
| | | | GAAGCGGAGCCGTTATGACCTGGGTGCGATCATG |
| | | | CGCCGAGCATGGGAGATGGCTCGGGAGGCGGCA |
| | | | TTCGCGGTTGGCGAGCGGGCACGGACTCACCTTG |
| | | | CTGCCGCGATGCGCAGCGCGTGGGCCGAAGCCA |
| | | | AGTTGGCACTCGCGCCCACGAAGACGGAGCAGG |
| | | | ATCGTCTCTCTCCGAGCGACATGATCGGACATGA |
| | | | GGACGCCTACCAAGGCCGGGTTCTAAAATAT |
| NLS0064 | ref1_3842117 | 72 | AAGATGGATACGACAAGCGCGATTACATTATTTG |
| | | | CGAAATAGATGGACAAATAAAAGACAAAGGACT |
| | | | GATGTATTTCCTTAAATCTGGACAAGTTGACCTCT |
| | | | TTCACATAGAAGTCACCACTCCCTTTGGGACAAT |
| | | | TTGGTGTCACGAAAACATAGAGGCCGAACTTCTT |
| | | | AGCTGAATTATCGCGCTCCGGGTTCTTATGCGGC |
| | | | TGAGTGAAGCGCGGGACAGCTTGCGAGCAGGGC |
| | | | CGCCAATGGCAGCCGGGATGACACAATGCTCGGT |
| | | | CTCCCGACGCTTCTTCAATCGGGAGCGCT |
| NLS0064 | ref1_3842278 | 73 | AGCTGAATTATCGCGCTCCGGGTTCTTATGCGGC |
| | | | TGAGTGAAGCGCGGGACAGCTTGCGAGCAGGGC |
| | | | CGCCAATGGCAGCCGGGATGACACAATGCTCGGT |
| | | | CTCCCGACGCTTCTTCAATCGGGAGCGCTTCGCA |
| | | | GCCCGGGGCGGCGCGCTCATGCGTCACGACCTGG |
| | | | GCCCTGCGCACCTTCGCGGCCCCGCCGTCCCGGC |
| | | | AGATCCCTGATGCCCCAAGTGGGCGGCCACTCCA |
| | | | TCAAAGAACCCCGGCCTGTGGCAGATCTCGTAGG |
| | | | CATACCGAGGTTCCGCAGTGCCCCCACC |

### REFERENCES

Altschul S.F., Gish W., Miller W., Myers E.W., Lipman D.J. (1990) Basic local alignment search tool. J. Mol. Biol. 215:403-410
Bentley D.R., Balasubramanian S., Swerdlow H.P., Smith G.P., Milton J., Brown D. G., Hall K.P., Evers D.J., Barnes C.L., Bignell H.R. et al. (2008) Accurate whole human genome sequencing using reversible terminator chemistry. Nature 456:53-59.
Caporaso J.G., Lauber C.L., Walters W.A., Berg-Lyons D., Huntley J., Fierer N., Owens S.M., Betley J., Fraser L., Bauer M., Gormley N., Gilbert J.A., Smith G., Knight R. (2012) Ultra-high-throughput microbial community analysis on the Illumina HiSeq and MiSeq platforms. ISME J 6:1621-1624.
Comai et al. (1983) An altered aroA gene product confers resistance to the herbicide glyphosate. Science 221(4608):370-371.
Green, P.N. 2005. Methylobacterium . In Brenner, D.J., N.R. Krieg, and J.T. Staley (eds.). “Bergey's Manual of Systematic Bacteriology. Volume two, The Proteobacteria. Part C, The alpha- , beta- , delta- , and epsilonproteobacteria.” Second edition. Springer, New York. Pages 567-571.
Green, P.N. 2006. Methylobacterium . In Dworkin, M., S. Falkow, E. Rosenberg, K.-H. Schleifer, and E. Stackebrandt (eds.). “The Prokaryotes. A Handbook on the Biology of Bacteria. Volume 5. Proteobacteria: Alpha and Beta Subclasses.” Third edition. Springer, New York. Pages 257-265.
Green and Ardley (2018) Review of the genus Methylobacterium and closely related organisms: a proposal that some Methylobacterium species be reclassified into a new genus, Methylorubrum gen. nov. International Journal of Systematic and Evolutionary Microbiology 68:2727-2748.
Konstantinos T. K. and Tiedje J.M. (2005) Proc. Nat. Acad. Sci. USA 102:2567-2572.
Lidstrom, M.E. 2006. Aerobic methylotrophic prokaryotes. In Dworkin, M., S. Falkow, E. Rosenberg, K.-H. Schleifer, and E. Stackebrandt (eds.). “The Prokaryotes. A Handbook on the Biology of Bacteria. Volume 2. Ecophysiology and biochemistry.” Third edition. Springer, New York. Pages 618-634.
Sy, A., Giraud, E., Jourand, P., Garcia, N., Willems, A., De Lajudie, P., Prin, Y., Neyra, M., Gillis, M., Boivin-Masson, C., and Dreyfus, B. 2001. Methylotrophic Methylobacterium Bacteria Nodulate and Fix Nitrogen in Symbiosis with Legumes. Jour. Bacteriol. 183(1):214-220.
Sanger F, Nicklen S, Coulson AR (1977) DNA sequencing with chain-terminating inhibitors. Proc Natl Acad Sci U S A. 74(12):5463-5467.
Vogel, H.J., and D.M. Bonner. 1956. Acetylornithinase of Escherichia coli: Partial purification and some properties. J. Biol. Chem. 218:97-106.
Whittenbury, R., S.L. Davies, and J.F. Wilkinson. 1970. Enrichment, isolation and some properties of methane-utilizing bacteria. J. Gen. Microbiol. 61:205-218.

Having illustrated and described the principles of the present disclosure, it should be apparent to persons skilled in the art that the invention can be modified in arrangement and detail without departing from such principles.

Although the materials and methods of this invention have been described in terms of various embodiments and illustrative examples, it will be apparent to those of skill in the art that variations can be applied to the materials and methods described herein without departing from the concept, spirit and scope of the invention. All such similar substitutes and modifications apparent to those skilled in the art are deemed to be within the spirit, scope and concept of the invention as defined by the appended claims.

1. A method for enhancing corn plant growth that comprises:
(a) applying a composition to a corn plant or a corn plant part, wherein the composition comprises Methylobacterium isolate selected from the group consisting of: NLS0648 (NRRL B-67741), NLS0648 (NRRL B-67741) and NLS0020 (NRRL B-50930), and NLS0020 (NRRL B-50930); wherein said composition further comprises at least one additional component selected from the group consisting of an additional active ingredient, an agriculturally acceptable adjuvant, and an agriculturally acceptable excipient; and,
(b) growing the corn plant to maturity, thereby enhancing growth of the corn plant.

2. The method of claim 1, wherein the composition is applied to a corn seed.

3. The method of claim 1, wherein the composition comprises a solid substance with the Methylobacterium grown thereon and adhered thereto, or an emulsion having the Methylobacterium grown therein.

4. The method of claim 1, wherein the composition comprises the Methylobacterium at a titer of about 1×10⁶ CFU/gm to about 1×10¹⁴ CFU/gm for a solid composition or at a titer of about 1×10⁶ CFU/mL to about 1×10¹¹ CFU/mL for a liquid composition.

5. The method of claim 1, wherein the composition comprises NLS0648 (NRRL B-67741).

6. The method of claim 5, wherein the composition further comprises NLS0109 (NRRL B-67340).

7. (canceled)

8. The method of claim 1, wherein the applied composition coats or partially coats the corn plant or a part thereof.

9. The method of claim 1, wherein the composition is applied to foliage of the corn plant.

10. The method of claim 9, wherein the additional active ingredient is a fungicide.

11. The method of claim 1, wherein the composition is applied at about the VE to about the V3 stage of development, about the V3 to about the V5 stage of development, about the V2 to V4, or V3 stage of development.

12.-13. (canceled)

14. The method of claim 1 wherein said composition is applied by spraying, coating, partially coating, immersing, and/or imbibing the corn plant or plant part with the composition.

15. The method of claim 14 wherein the applied composition coats or partially coats the corn plant or a part thereof, wherein partial coating includes coating at least 5%, 10%, 20%, 30%, 40%, 50%, 60%, 70%, 80%, 90%, 95%, 98%, 99%, or about 99.5% of the surface area of the corn plant or a part thereof.

16. The method of claim 15, wherein said corn plant part is a seed.

17.-18. (canceled)

19. The method of claim 1, wherein the additional active ingredient is selected from the group consisting of a fungicide, insecticide, nematicide, and a second biological.

20. The method of claim 19, wherein the additional active ingredient is a second biological selected from the group consisting of: other beneficial microorganisms, microbial extracts, natural products, plant growth activators or a plant defense agent.

21. The method of claim 19, wherein the additional active ingredient is selected from the group consisting of clothianidin, Bacillus firmus, abamectin, thiamethoxam, imidacloprid, azoxystrobin, fluopyram, fluoxastrobin, ipconazole, mefenoxam, metalaxyl, penflufen, prothioconazole, pyraclostrobin, and sedaxane.

22. The method of claim 1, wherein the additional active ingredient is selected from the group consisting of the Methylobacterium isolates in Table 1.

23. The method of claim 1, wherein the additional active ingredient is selected from the group consisting of: NLS0017 (NRRL B-50931), NLS0021 (NRRL B-50939), NLS0037 (NRRL B-50941), NLS0038 (NRRL B-50942), NLS0042 (NRRL B-50932), NLS0046 (NRRL B-50929), NLS0062 (NRRL B-50937), NLS0064 (NRRL B-50938), NLS0065 (NRRL B-50935), NLS0066 (NRRL B-50940), NLS0068 (NRRL B-50934), NLS0069 (NRRL B-50936), NLS0089 (NRRL B-50933), NLS0109 (NRRL B-67742), and NLS0807 (NRRL B-67743).

24.-45. (canceled)

46. A method for activating plant growth comprising:
(i) applying a composition to a plant or a plant part, wherein the composition comprises (i) Methylobacterium isolate selected from the group consisting of: NLS0648 (NRRL B-67741), NLS0648 (NRRL B-67741) and NLS0020 (NRRL B-50930), and NLS0020 (NRRL B-50930), and (ii) at least one additional component selected from the group consisting of an additional active ingredient, an agriculturally acceptable adjuvant, and an agriculturally acceptable excipient; wherein the composition is a plant growth activator.

47. A composition for a plant growth growth activator comprising:
(i) a Methylobacterium isolate selected from the group consisting of: NLS0648 (NRRL B-67741), NLS0648 (NRRL B-67741) and NLS0020 (NRRL B-50930), and NLS0020 (NRRL B-50930), and
(ii) at least one additional component selected from the group consisting of an additional active ingredient, an agriculturally acceptable adjuvant, and an agriculturally acceptable excipient;
wherein the composition is a plant growth activator.
